(19) United States
(12) Patent Application Publication
(10) Pub. No.: US 2022/0186194 A1
(43) Pub. Date: Jun. 16, 2022

(54) METHODS OF PURIFYING ADENOVIRUS

(71) Applicant: ASTRAZENECA UK LIMITED, CAMBRIDGE, MA (US)

(72) Inventors: THOMAS LINKE, GAITHERSBURG, MD (US); ADRIAN VICTOR ALLEN JOSEPH, GAITHERSBURG, MD (US); MATTHEW THOMAS ASPELUND, GAITHERSBURG, MD (US); JESSICA PRENTICE, GAITHERSBURG, MD (US)

(21) Appl. No.: 17/547,317

(22) Filed: Dec. 10, 2021

Related U.S. Application Data
(60) Provisional application No. 63/123,564, filed on Dec. 10, 2020.

(51) Int. Cl. C12N 7/02; B01D 15/36; B01D 15/12; B01D 15/18

(52) U.S. Cl. CPC C12N 7/02; B01D 15/363; C12N 2710/10351; B01D 15/1871; C12N 2710/10051; B01D 15/125

(57) ABSTRACT

Methods of purifying adenovirus that can be performed on a large scale. The methods purify adenovirus from an adenovirus-containing sample comprising or derived from a host cell population by clarifying the sample, wherein clarification comprises depth filtration followed by microfiltration; processing the clarified sample by anion exchange chromatography; and processing the anion exchange product by tangential flow filtration (TFF) to provide a TFF product.

### FIELD OF THE INVENTION

The present invention relates to methods of purifying adenovirus. More particularly, the invention relates to methods of purifying adenovirus that can be performed on a large scale.

### BACKGROUND

Adenoviruses are double-stranded DNA viruses with a genome of approximately 26-46 kb. Adenoviruses are species-specific and different serotypes have been isolated from a variety of mammalian species. Human adenoviruses are ubiquitous, and most people have been infected with one or more serotypes, leading to lifelong immunity.

Modified adenoviruses can be used as vectors to deliver DNA coding for foreign antigens. Replication-deficient adenovirus vectors have been employed extensively for vaccines because they induce a strong humoral and T cell response to the heterologous gene encoded by the vector.

The production process for adenovirus vectors involves infecting host cells with the adenovirus and culturing the host cells to increase the virus titer. The cells are then lysed before downstream purification treatment to remove impurities including cells and cell debris.

Current methods for purification of adenoviruses are uneconomical and lack scalability. There therefore exists a need for the development of a large-scale process for purifying adenoviruses generated within host cell culture systems.

### SUMMARY OF THE INVENTION

The present invention relates, at least in part, to the development of improved adenovirus purification methods that effectively purify adenovirus from host cell culture and have reduced processing time compared to alternative purification methods. The purification methods of the present invention may also have reduced reliance on certain raw materials that can be in short supply compared to alternative purification methods. The methods of the present invention may therefore have particular utility where large quantities of adenovirus vectors are required, such as for the provision of adenovirus-based vaccines for epidemic and pandemic diseases.

Accordingly, in one aspect, there is provided a method of purifying adenovirus from an adenovirus-containing sample comprising or derived from a host cell population having a cell density of at least about 4×10⁶ cells/mL, the method comprising:
(a) clarifying the sample to provide a clarified sample, wherein clarification comprises depth filtration followed by microfiltration;
(b) processing the clarified sample by anion exchange chromatography to provide an anion exchange product; and
   processing the anion exchange product by tangential flow filtration (TFF) to provide a TFF product, wherein TFF comprises ultrafiltration and diafiltration.

In a further aspect, there is provided a method of purifying adenovirus as described in FIG. 2.

In a yet further aspect, there is provided a method of purifying adenovirus as described in Example 2.

In a yet further aspect, there is provided a purified adenovirus obtainable by or obtained by a method of the invention.

In a yet further aspect, there is provided a drug substance obtainable by or obtained by a method of the invention.

Aspects and embodiments of the invention are set out in the appended claims. These and other aspects and embodiments of the invention are also described herein.

### BRIEF DESCRIPTION OF THE FIGURES

FIG. 1 shows an exemplary adenovirus purification process comprising the steps of cell lysis and nuclease digestion of the bioreactor product, clarification, first tangential flow filtration, anion exchange chromatography, second tangential flow filtration and sterile filtration.

FIG. 2 shows an exemplary adenovirus purification process according to the invention comprising the steps of cell lysis and nuclease digestion of the starting material, clarification, anion exchange chromatography, tangential flow filtration, formulation and sterile filtration.

FIG. 3 shows a variation of the exemplary adenovirus purification process according to the invention comprising the additional step of mixed mode size exclusion chromatography.

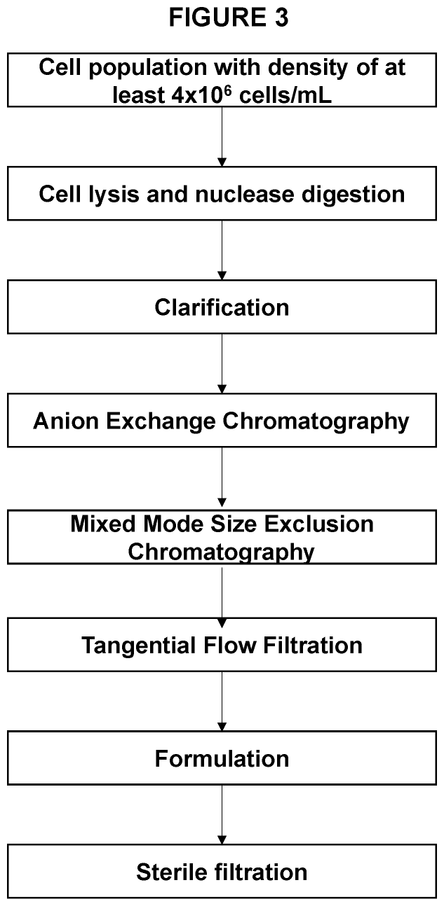

### BRIEF DESCRIPTION OF THE SEQUENCE LISTING

| SEQ | | |
|---|---|---|
| ID | | |
| NO: | Description | Sequence |
| 1 | Amino acid | MFVFLVLLPL VSSQCVNLTT RTQLPPAYTN |
| | sequence of | SFTRGVYYPD KVFRSSVLHS TQDLFLPFFS |
| | the spike | NVTWFHAIHV SGTNGTKRFD NPVLPFNDGV |
| | protein of | YFASTEKSNI IRGWIFGTTL DSKTQSLLIV |
| | the | NNATNVVIKV CEFQFCNDPF LGVYYHKNNK |
| | SARS-CoV-2 | SWMESEFRVY SSANNCTFEY VSQPFLMDLE |
| | strain of | GKQGNFKNLR EFVFKNIDGY FKIYSKHTPI |
| | the SARS-CoV | NLVRDLPQGF SALEPLVDLP IGINITRFQT |
| | species of | LLALHRSYLT PGDSSSGWTA GAAAYYVGYL |
| | coronavirus | QPRTFLLKYN ENGTITDAVD CALDPLSETK |
| | | CTLKSFTVEK GIYQTSNFRV QPTESIVRFP |
| | | NITNLCPFGE VFNATRFASV YAWNRKRISN |
| | | CVADYSVLYN SASFSTFKCY GVSPTKLNDL |
| | | CFTNVYADSF VIRGDEVRQI APGQTGKIAD |
| | | YNYKLPDDFT GCVIAWNSNN LDSKVGGNYN |
| | | YLYRLFRKSN LKPFERDIST EIYQAGSTPC |
| | | NGVEGFNCYF PLQSYGFQPT NGVGYQPYRV |
| | | VVLSFELLHA PATVCGPKKS TNLVKNKCVN |
| | | FNFNGLTGTG VLTESNKKFL PFQQFGRDIA |
| | | DTTDAVRDPQ TLEILDITPC SFGGVSVITP |
| | | GTNTSNQVAV LYQDVNCTEV PVAIHADQLT |
| | | PTWRVYSTGS NVFQTRAGCL IGAEHVNNSY |
| | | ECDIPIGAGI CASYQTQTNS PRRARSVASQ |
| | | SIIAYTMSLG AENSVAYSNN SIAIPTNFTI |
| | | SVTTEILPVS MTKTSVDCTM YICGDSTECS |
| | | NLLLQYGSFC TQLNRALTGI AVEQDKNTQE |
| | | VFAQVKQIYK TPPIKDFGGF NFSQILPDPS |
| | | KPSKRSFIED LLFNKVTLAD AGFIKQYGDC |
| | | LGDIAARDLI CAQKFNGLTV LPPLLTDEMI |
| | | AQYTSALLAG TITSGWTFGA GAALQIPFAM |
| | | QMAYRFNGIG VTQNVLYENQ KLIANQFNSA |
| | | IGKIQDSLSS TASALGKLQD VVNQNAQALN |
| | | TLVKQLSSNF GAISSVLNDI LSRLDKVEAE |
| | | VQIDRLITGR LQSLQTYVTQ QLIRAAEIRA |
| | | SANLAATKMS ECVLGQSKRV DFCGKGYHLM |
| | | SFPQSAPHGV VFLHVTYVPA QEKNFTTAPA |
| | | ICHDGKAHFP REGVFVSNGT HWFVTQRNFY |
| | | EPQIITTDNT FVSGNCDVVI GIVNNTVYDP |
| | | LQPELDSFKE ELDKYFKNHT SPDVDLGDIS |
| | | GINASVVNIQ KEIDRLNEVA KNLNESLIDL |
| | | QELGKYEQYI KWPWYIWLGF IAGLIAIVMV |
| | | TIMLCCMTSC CSCLKGCCSC GSCCKFDEDD |
| | | SEPVLKGVKL HYT |
| 2 | Amino acid | MDAMKRGLCC VLLLCGAVFV SASQEIHARF |
| | sequence of | RRFVFLVLLP LVSSQCVNLT TRTQLPPAYT |
| | the spike | NSFTRGVYYP DKVFRSSVLH STQDLFLPFF |
| | protein of | SNVTWFHAIH VSGTNGTKRF DNPVLPFNDG |
| | the | VYFASTEKSN IIRGWIFGTT LDSKTQSLLI |
| | SARS-CoV-2 | VNNATNVVIK VCEFQFCNDP FLGVYYHKNN |
| | strain of | KSWMESEFRV YSSANNCTFE YVSQPFLMDL |
| | the SARS-CoV | EGKQGNFKNL REFVFKNIDG YFKIYSKHTP |
| | species of | INLVRDLPQG FSALEPLVDL PIGINITRFQ |
| | coronavirus | TLLALHRSYL TPGDSSSGWT AGAAAYYVGY |
| | with the | LQPRTFLLKY NENGTITDAV DCALDPLSET |
| | signal | KCTLKSFTVE KGIYQTSNFR VQPTESIVRF |
| | peptide of | PNITNLCPFG EVFNATRFAS VYAWNRKRIS |
| | the human | NCVADYSVLY NSASFSTFKC YGVSPTKLND |
| | tissue | LCFTNVYADS FVIRGDEVRQ IAPGQTGKIA |
| | plasminogen | DYNYKLPDDF TGCVIAWNSN NLDSKVGGNY |
| | activator | NYLYRLFRKS NLKPFERDIS TEIYQAGSTP |
| | gene (tPA) | CNGVEGFNCY FPLQSYGFQP TNGVGYQPYR |
| | at the | VVVLSFELLH APATVCGPKK STNLVKNKCV |
| | N-terminus | NFNFNGLTGT GVLTESNKKF LPFQQFGRDI |
| | | ADTTDAVRDP QTLEILDITP CSFGGVSVIT |
| | | PGTNTSNQVA VLYQDVNCTE VPVAIHADQL |
| | | TPTWRVYSTG SNVFQTRAGC LIGAEHVNNS |
| | | YECDIPIGAG ICASYQTQTN SPRRARSVAS |
| | | QSIIAYTMSL GAENSVAYSN NSIAIPTNFT |
| | | ISVTTEILPV SMTKTSVDCT MYICGDSTEC |
| | | SNLLLQYGSF CTQLNRALTG IAVEQDKNTQ |
| | | EVFAQVKQIY KTPPIKDFGG FNFSQILPDP |
| | | SKPSKRSFIE DLLFNKVTLA DAGFIKQYGD |
| | | CLGDIAARDL ICAQKFNGLT VLPPLLTDEM |
| | | IAQYTSALLA GTITSGWTFG AGAALQIPFA |
| | | MQMAYRFNGI GVTQNVLYEN QKLIANQFNS |
| | | AIGKIQDSLS STASALGKLQ DVVNQNAQAL |
| | | NTLVKQLSSN FGAISSVLND ILSRLDKVEA |
| | | EVQIDRLITG RLQSLQTYVT QQLIRAAEIR |
| | | ASANLAATKM SECVLGQSKR VDFCGKGYHL |
| | | MSFPQSAPHG VVFLHVTYVP AQEKNFTTAP |
| | | AICHDGKAHF PREGVFVSNG THWFVTQRNF |
| | | YEPQIITTDN TFVSGNCDVV IGIVNNTVYD |
| | | PLQPELDSFK EELDKYFKNH TSPDVDLGDI |
| | | SGINASVVNI QKEIDRLNEV AKNLNESLID |
| | | LQELGKYEQY IKWPWYIWLG FIAGLIAIVM |
| | | VTIMLCCMTS CCSCLKGCCS CGSCCKFDED |
| | | DSEPVLKGVK LHYT |

### DETAILED DESCRIPTION OF THE INVENTION

### Adenovirus-Containing Sample

The methods of the present invention are capable of purifying adenovirus from an adenovirus-containing sample on a large scale. For example, the methods of the present invention may be capable of processing volumes up to about 5000 litres, e.g. from about 3 litres to about 3000 litres, preferably in the range of about 200 litres up to about 2000 litres.

The adenovirus-containing sample may comprise at least one host cell protein (HCP). As used herein, the term “HCP” refers to proteins produced or encoded by a host cell population.

The adenovirus-containing sample may have a HCP concentration of at least about 20,000 ng/mL, at least about 30,000 ng/mL, at least about 40,000 ng/mL, at least about 50,000 ng/mL, at least about 60,000 ng/mL, at least about 70,000 ng/mL, at least about 80,000 ng/mL, at least about 90,000 ng/mL or at least about 100,000 ng/mL. In preferred embodiments, the adenovirus-containing sample has a HCP concentration of at least about 50,000 ng/mL.

The adenovirus-containing sample may have a HCP concentration of up to about 100,000 ng/mL, up to about 90,000 ng/mL, up to about 80,000 ng/mL, up to about 70,000 ng/mL, up to about 60,000 ng/mL, up to about 50,000 ng/mL, up to about 40,000 ng/mL, up to about 30,000 ng/mL, or up to about 20,000 ng/mL.

The adenovirus-containing sample may have a HCP concentration of between about 20,000 ng/mL and about 100,000 ng/mL, between about 30,000 ng/mL and about 90,000 ng/mL or between about 50,000 ng/mL and about 80,000 ng/mL.

Any upstream virus production process known in the art that can be adapted to large scale cell culture of host cells (e.g. mammalian host cells) may be utilized to generate the starting material for the methods of the present invention.

In preferred embodiments, the adenovirus-containing sample comprises or consists of a host cell population. The host cell population may be cultured in a cell culture vessel. As used herein, a “cell culture vessel” refers to a container suitable for culturing cells. Preferably, the cell culture vessel is a bioreactor. As used herein “bioreactor” means a cell culture vessel adapted for a large scale process. For example, in some embodiments, the bioreactor has a capacity of at least about 1 L, preferably at least about 1.2 L, about 3 L, about 50 L, about 1000 L, about 2000 L, about 3000 L, or about 5000 L, most preferably at least about 2000 L. In some embodiments, the bioreactor has a capacity of at least about 7×10⁹ viable T-REx™ cells, preferably at least about 2.1×10¹⁰ viable T-REx™ cells, at least about 3.5×10¹¹ viable T-REx™ cells, at least about 5×10¹² viable T-REx™ cells or at least about 3×10¹³ viable T-REx™ cells, most preferably at least about 5×10¹² viable T-REx™ cells.

The host cell population may have a cell density (e.g. viable cell density) at time of harvest of at least at least about 5×10⁶ cells/mL, at least about 6×10⁶ cells/mL, at least about 7×10⁶ cells/mL, at least about 8×10⁶ cells/mL, at least about 9×10⁶ cells/mL or at least about 1×10⁷ cells/mL. In preferred embodiments, the host cell population has a cell density (e.g. viable cell density) at time of harvest of at least about 4×10⁶ cells/mL.

The host cell population may have a cell density (e.g. viable cell density) at time of harvest of up to about 1×10⁹ cells/mL, up to about 1×10⁸ cells/mL, up to about 8×10⁷ cells/mL, up to about 6×10⁷ cells/mL, up to about 4×10⁷ cells/mL, up to about 2×10⁷ cells/mL, up to about 1×10⁷ cells/mL, up to about 8×10⁶ cells/mL or up to about 6×10⁶ cells/mL. In some embodiments, the host cell population has a cell density (e.g. viable cell density) at time of harvest of up to about 8×10⁶ cells/mL. In preferred embodiments, the host cell population has a cell density (e.g. viable cell density) at time of harvest of up to about 1×10⁷ cells/mL.

The host cell population may have a cell density (e.g. viable cell density) at time of harvest of between about 4×10⁶ cells/mL and about 1×10⁹ cells/mL, between about 4×10⁶ cells/mL and about 1×10⁸ cells/mL or between about 4×10⁶ cells/mL and about 1×10⁷ cells/mL.

In preferred embodiments, the methods of the present invention are capable of processing host cell culture volumes as disclosed herein, i.e. greater than 200 litres (e.g. about 2000 litres) and having a cell density (e.g. viable cell density) at time of harvest at disclosed herein (e.g. of at least about 4×10⁶ cells/mL).

In preferred embodiments, the methods of the present invention are capable of processing a host cell population having a cell density (e.g. viable cell density) at time of harvest as set forth above and a HCP concentration as set forth above. For example, the host cell population may have a cell density of at least about 4×10⁶ cells/mL and a HCP concentration of at least about 50,000 ng/mL.

### Cell Lysis and Nuclease Treatment

Following culture of adenovirus-containing host cell population, the host cells are lysed to release intracellular adenovirus. The lysis step may also provide for a potential to inactivate potential adventitious agents (in particular, enveloped viruses such as herpes viruses or retroviruses) which could hypothetically contaminate the cell culture at a low level.

Accordingly, in some embodiments the methods of the invention comprise a cell lysis step. Methods that can be used for cell lysis are known in the art, and include both non-mechanical lysis methods (such as detergent lysis, enzyme treatment, hypertonic and/or hypotonic lysis) and mechanical methods (such as freeze-thaw, solid shear, liquid shear, sonication and high pressure extrusion).

In preferred embodiments, the host cells are lysed using a cell lysis agent (e.g. a detergent). Use of a detergent for cell lysis has the advantage that it is straightforward to implement, and that it is easily scalable. Detergents that can be used for cell lysis are known in the art. Detergents used for cell lysis in the methods of the present invention can include but are not limited to anionic, cationic, zwitterionic, and nonionic detergents. In preferred embodiments, the detergent is a nonionic detergent. Examples of suitable nonionic detergents include Polysorbate (e.g. Polysorbate-20 or Polysorbate-80) and Triton (e.g. Triton-X). In one embodiment, the nonionic detergent is Polysorbate-20. The optimal concentration of the nonionic detergent used to lyse the host cell population may vary, for instance within the range of about 0.005-0.025 kg detergent/kg vessel, about 0.01-0.02 kg detergent/kg cell culture vessel, or about 0.011-0.016 kg detergent/kg cell culture vessel. As used herein, “kg cell culture vessel” means the total mass of the host cell population and the cell culture medium in the cell culture vessel. In preferred embodiments, the concentration of the nonionic detergent (e.g. Polysorbate-20) used to lyse the host cell population is about 0.013 kg detergent/kg cell culture vessel. The host cells may be incubated with the nonionic detergent (e.g. Polysorbate-20) for sufficient time for all or substantially all of the cells in the host cell population to be lysed. In embodiments, the host cells are incubated with the nonionic detergent (e.g. Polysorbate-20) for at least about 15 minutes prior to a nuclease treatment step. In embodiments, the host cells are incubated with the nonionic detergent (e.g. Polysorbate-20) for up to 30 minutes prior to a nuclease treatment step. In embodiments, the host cells are not incubated with the nonionic detergent (e.g. Polysorbate-20) for longer than 30 minutes prior to a nuclease treatment step.

In embodiments, the detergent (e.g. Polysorbate-20) forms part of a lysis buffer. Accordingly, in some embodiments, the host cells are lysed using a lysis buffer comprising at least one detergent (e.g. Polysorbate-20). An exemplary lysis buffer that may be used in the methods of the invention comprises about 500 mM tris, about 20 mM MgCl₂, about 50% (w/v) sucrose and about 10% (v/v) Polysorbate 20, and has a pH of about 8. The optimal concentration of the lysis buffer used to lyse the cell population may vary, for instance within the range of about 0.05-0.25 kg lysis buffer/kg cell culture vessel, about 0.10-0.20 kg lysis buffer/kg cell culture vessel, or about 0.11-0.16 kg lysis buffer/kg cell culture vessel. In preferred embodiments, the concentration of the lysis buffer is about 0.13 kg lysis buffer/kg cell culture vessel.

Autolysis of the infected host cells by the adenovirus in the host cells may also provide for substantial release of intracellular adenovirus and may be used in the methods of the invention.

While the cell lysis step significantly increases the yield of adenovirus from the host cell population, it also results in release of contaminants such as intracellular proteins and cellular genomic nucleic acids. The presence of nucleic acid may be a particular concern for adenovirus, as DNA is known to mediate virus particle aggregation.

The methods of the present invention may therefore comprise a nuclease-treatment step following cell lysis. Alternatively the nuclease step might be conducted simultaneously to the lysis by adding the nuclease to the cell prior to lysis. Nucleases that are suitable for use in the methods of the invention include DNases and RNases, including non-specific DNA and RNA endonucleases, such as Benzonase® (Merck). In preferred embodiments, Benzonase® is added to the lysed cells. The addition of nuclease (e.g. Benzonase®) may reduce nucleic acid chain length, which facilitates its removal in later steps, reduce viscosity and assists in the reduction of nucleic acid-mediated aggregation. The nuclease may be added in an amount to achieve release of acceptable levels of adenovirus and sufficient reduction or treatment of cellular genomic acid. For example, Benzonase® may be added to the lysed cells to achieve a final concentration of about 5,000 Units/kg lysate to 25,000 Units/kg lysate, about 10,000 Units/kg lysate to 20,000 Units/kg lysate, or about 15,000 Units/kg lysate. In preferred embodiments, the final concentration of Benzonase® is about 15,000 Units/kg lysate.

The cell lysis and nuclease-treatment may be carried out for sufficient time to achieve release of acceptable levels of adenovirus and sufficient reduction or treatment of cellular genomic acid. The cell lysis and nuclease-treatment may be carried out for at last 1 hour, at least 2 hours, at least 3 hours, at least 4 hours, or at least 5 hours. In preferred embodiments, the cell lysis and nuclease treatment is carried out for at least 2 hours.

The optimum temperature for cell lysis and nuclease treatment may be determined by a person skilled in the art. In preferred embodiments, the cell lysis and nuclease treatment is carried out at a temperature of from about 27° C. to about 40° C., preferably from about 31° C. to about 35° C., most preferably about 33° C.

The resulting product following cell lysis and nuclease treatment is nuclease-treated cell lysate.

### Clarification

The methods of the present invention comprise a clarification step. The clarification step seeks to remove impurities including cell debris from the adenovirus-containing sample (e.g. nuclease-treated cell lysate).

The nuclease-treated cell lysate may have been derived from a host cell population having a cell density (e.g. viable cell density) at time of harvest at least about 4×10⁶ cells/mL, at least about 5×10⁶ cells/mL, at least about 6×10⁶ cells/mL, at least about 7×10⁶ cells/mL, at least about 8×10⁶ cells/mL, at least about 9×10⁶ cells/mL or at least about 1×10⁷ cells/mL. In preferred embodiments, the nuclease-treated cell lysate was derived from a host cell population having a cell density (e.g. viable cell density) at time of harvest of at least about 4×10⁶ cells/mL.

The nuclease-treated cell lysate may have been derived from a host cell population having a cell density (e.g. viable cell density) at time of harvest of up to about 1×10⁹ cells/mL, up to about 1×10⁸ cells/mL, up to about 8×10⁷ cells/mL, up to about 6×10⁷ cells/mL, up to about 4×10⁷ cells/mL, up to about 2×10⁷ cells/mL, up to about 1×10⁷ cells/mL, up to about 8×10⁶ cells/mL or up to about 6×10⁶ cells/mL. In preferred embodiments, the nuclease-treated cell lysate was derived from a cell culture having a cell density (e.g. viable cell density) at time of harvest of up to about 8×10⁶ cells/mL.

The nuclease-treated cell lysate may have been derived from a host cell population having a cell density (e.g. viable cell density) at time of harvest of between about 4×10⁶ cells/mL and about 1×10⁹ cells/mL, between about 4×10⁶ cells/mL and about 1×10⁸ cells/mL or between about 4×10⁶ cells/mL and about 1×10⁷ cells/mL.

The nuclease-treated cell lysate may have a HCP concentration of at least about 20,000 ng/mL, at least about 30,000 ng/mL, at least about 40,000 ng/mL, at least about 50,000 ng/mL, at least about 60,000 ng/mL, at least about 70,000 ng/mL, at least about 80,000 ng/mL, at least about 90,000 ng/mL or at least about 100,000 ng/mL. In preferred embodiments, the nuclease-treated cell lysate has a HCP concentration of at least about 50,000 ng/mL.

The nuclease-treated cell lysate may have a HCP concentration of up to about 100,000 ng/mL, up to about 90,000 ng/mL, up to about 80,000 ng/mL, up to about 70,000 ng/mL, up to about 60,000 ng/mL, up to about 50,000 ng/mL, up to about 40,000 ng/mL, up to about 30,000 ng/mL, or up to about 20,000 ng/mL.

The nuclease-treated cell lysate may have a HCP concentration of between about 20,000 ng/mL and about 100,000 ng/mL, between about 30,000 ng/mL and about 90,000 ng/mL or between about 50,000 ng/mL and about 80,000 ng/mL.

In preferred embodiments, the input material for the clarification step is nuclease-treated cell lysate derived from a host cell population having a cell density (e.g. viable cell density) at time of harvest as set forth above and the cell lysate having a HCP concentration as set forth above. For example, the nuclease-treated cell lysate may have been derived from a host cell population having a cell density of at least about 4×10⁶ cells/mL and may have a HCP concentration of at least about 50,000 ng/mL. The nuclease-treated cell lysate may have been obtained as outlined in the above Cell lysis and nuclease treatment section.

The clarification step comprises subjecting the adenovirus-containing sample (e.g. nuclease-treated cell lysate) to depth filtration, to provide a filtrated sample. Depth filtration refers to a method of removing particles from solution using one or more depth filters. A depth filter comprises a three-dimensional matrix that creates a maze-like path through which the sample passes. The principle retention mechanisms of depth filters rely on random adsorption and mechanical entrapment throughout the depth of the matrix.

In various embodiments, the depth filter comprises filter membranes or sheets of wound cotton, polypropylene, rayon cellulose, silica, fiberglass, sintered metal, porcelain, diatomaceous earth, or other known components. In preferred embodiments, the depth filter comprises polypropylene (e.g. glass fibre-reinforced polypropylene) and cellulose, and optionally diatomaceous earth.

Suitable depth filters for the clarification step of the methods of the invention include depth filters having a nominal filter rating of about 9 μm, about 8 μm, about 7 μm, about 6 μm, about 5 μm, about 4 μm about 3 μm, about 2 μm, about 1 μm, and/or about 0.1 μm. In some embodiments, the depth filter has a nominal filter rating of between about 0.2 μm and about 2 μm. In some embodiments, the depth filter has a nominal filter rating of up to about 0.1 μm.

Suitable depth filters for the clarification step of the methods of the invention include a Millistak+® HC Pro Pod depth filter, COSP media, Millistak+® Pod depth filter, XOHC media and Millistak+® HC Pro Pod depth filter, XOSP media (all Millipore, available through Sigma-Aldrich).

The clarification step may involve the use of multiple different types of depth filters, e.g. having different nominal filter ratings. In some embodiments, two or more different types of depth filters are used. In some embodiments, three or more different types of depth filters are used. The depth filters may be arranged in series or in parallel, preferably in series.

The present inventors have observed that the use of two different types of depth filters in series (e.g. a depth filter having a nominal filter rating of about 0.2 μm to about 2 μm, such as Millistak+® HC Pro Pod depth filter, COSP media followed by a depth filter having a nominal filter rating of up to about 0.1 μm, such as Millistak+® Pod depth filter, XOHC media or Millistak+® HC Pro Pod depth filter, XOSP media) may assist in the capture of impurities and thereby better protect the membrane in the subsequent microfiltration step from fouling. The use of two different depth filters in series may also assist in reducing the quantities of the first depth filter (e.g. Millistak+® HC Pro Pod COSP filter) required as compared with when only a single type of depth filter is used (e.g. Millistak+® HC Pro Pod COSP filter).

In preferred embodiments, the clarification step involves the use of two different types of depth filters (e.g. depth filters having different nominal filter ratings) arranged in series. The two different types of depth filter may be any combination of the following depth filters: Millistak+® HC Pro Pod depth filter, COSP media; Millistak+® Pod depth filter, XOHC media; and Millistak+® HC Pro Pod depth filter, XOSP media. The two different types of depth filters may be arranged in accordance with their nominal filter rating, e.g. a depth filter having a nominal filter rating of about 0.2 μm to about 2 μm followed by a depth filter having a nominal filter rating of up to about 0.1 μm. In some embodiments, the two different types of depth filters are Millistak+® HC Pro Pod depth filter, COSP media, and Millistak+® HC Pro Pod depth filter, XOSP media. In some embodiments, the two different types of depth filters are Millistak+® HC Pro Pod COSP filter and Millistak+® Pod XOHC filter. In particularly preferred embodiments, the clarification step involves the use of a Millistak+® HC Pro Pod COSP filter and a Millistak+® Pod XOHC filter connected in series. In embodiments in which the clarification step involves the use of two different types of depth filters arranged in series, the depth filters may be loaded in a ratio of 3 first depth filter:1 second depth filter, e.g. 3 Millistak+® HC Pro Pod COSP filters:1 Millistak+® Pod XOHC filter.

Alternatively, the clarification step may involve the use of a single type of depth filter. In embodiments in which a single type of depth filter is used, the depth filter may have a nominal filter rating of about 0.2 μm to about 2 μm, e.g. a Millistak+® HC Pro Pod depth filter, COSP media.

The present inventors have shown that the three filter train series of: Millistak+® HC Pro Pod COSP filter followed by microfiltration; Millistak+® HC Pro Pod COSP filter followed by Millistak+® HC Pro Pod depth filter, XOSP media followed by microfiltration; and Millistak+® HC Pro Pod COSP filter followed by Millistak+® Pod XOHC filter followed by microfiltration have comparable performance in terms of yield and quality of the product obtained from the subsequent anion exchange step.

Prior to loading of the adenovirus-containing sample (e.g. nuclease-treated cell lysate), the depth filters may be equilibrated with an equilibration buffer. An exemplary equilibration buffer that may be used in the methods of the invention comprises about 50 mM tris, about 2 mM MgCl₂, about 5% (w/v) sucrose and about 1% (v/v) Polysorbate 20, and has a pH of about 8. The optimum volumetric throughput during the equilibration step may be at least about 15 L/m², preferably at least about 20 L/m², at least about 25 L/m² or at least about 30 L/m², most preferably at least about 25 L/m².

After depth filtration, the filtrated sample undergoes microfiltration. During microfiltration, the filtrated sample is passed through a microfiltration membrane. The particular microfiltration membrane selected will have pores of a size sufficiently large for adenovirus to pass through but small enough to clear impurities (e.g. partially lysed cells, cell debris and/or aggregates). In some embodiments, the microfiltration membrane has a membrane pore size of less than about 1 μm, less than about 0.75 μm, less than about 0.5 μm, or about less than about 0.25 μm. In preferred embodiments, the microfiltration membrane has a pore size of about 0.2 μm.

Suitable membrane materials for microfiltration may include regenerated cellulose, polyethersulfone, polysulfone, or derivatives thereof, preferably polyethersulfone. In preferred embodiments, the microfiltration membrane comprises polyethersulfone and has a pore size of about 0.2 μm. A suitable microfiltration membrane for use in the methods of the present invention is the Millipore Express® SHC 0.5/0.2 μm filter.

After loading the filtrated sample, chase buffer may be added to the microfiltration membrane. An exemplary chase buffer that may be used in the methods of the invention comprises about 50 mM tris, about 2 mM MgCl₂, about 5% (w/v) sucrose and about 1% (v/v) Polysorbate-20, and has a pH 8.

The optimum volumetric throughput during the chase step may be from about 15 L/m² to about 40 L/m², preferably from about 20 L/m² to about 40 L/m², at least about 25 L/m² to about 40 L/m² or at least about 30 L/m² to about 40 L/m², most preferably at least about 25 L/m² to about 40 L/m².

The optimum temperature for the clarification step may be determined by a person skilled in the art. In preferred embodiments, the clarification step is carried out at a temperature of from about 15° C. to about 35° C., more preferably from about 15° C. to about 27° C.

After clarification, the sample may be tested for various parameters. For example, in some embodiments, the clarified sample has a pH of from about 7 to about 9, preferably from about 7.5 to about 8.5. In some embodiments, the clarified sample has a conductivity at 25° C. of less than about 35 mS/cm, less than about 30 mS/cm, preferably less than about 25 mS/cm. Accordingly, in some embodiments, the clarified sample has a pH of from about 7.5 to about 8.5 and a conductivity at 25° C. of less than about 25 mS/cm.

### Anion Exchange Chromatography

Following clarification, the methods of the invention comprise an anion exchange chromatography step to remove process-related impurities from the clarified sample (e.g. clarified lysate). During the anion exchange chromatography step, adenovirus particles are bound to a positively charged material, e.g. a membrane, cartridge or column, and subsequent elution allows for separating the adenovirus particles from impurities.

Purification methods known in the art may comprise a concentration step (e.g. tangential flow filtration, TFF) after clarification and before anion exchange chromatography to reduce the bulk volume and remove small molecules such as small proteins that interact with the virus (Jort Vellinga, J. Patrick Smith, Agnieszka Lipiec, Dragomira Majhen, Angelique Lemckert, Mark van Ooij, Paul Ives, Christopher Yallop, Jerome Custers, and Menzo Havenga. Human Gene Therapy. Apr 2014.318-327).

The methods of the present invention are characterized in that the clarified sample (e.g. clarified lysate) is processed by anion exchange chromatography without any intervening concentration steps such as TFF. Elimination of an intervening concentration step (e.g. TFF) results in significant process simplification and reduced raw material consumption and eliminates a large volume of waste stream. The elimination of an intervening concentration step (e.g. TFF) between clarification and anion exchange chromatography can drastically reduce processing time; in the methods of the present invention, removal of this step shortened processing time by one day. Thus, the methods of the present invention have improved scalability and throughput but still provide acceptable product quality (as shown in Example 3).

Anionic exchange substituents may be attached to matrices in order to form anionic supports for chromatography. Non-limiting examples of anionic exchange substituents that may be used in the methods of the invention include diethylaminoethyl (DEAF), quaternary aminoethyl (QAE) and quaternary ammonium (Q) groups. Preferably, the anion exchange material comprises DEAF or Q anionic exchange substituents.

In preferred embodiments, the anion exchange material comprises an anion exchange membrane. Anion exchange membranes are thin, synthetic membranes carrying anionic exchange substituents capable of interacting with at least one substance in contact within a fluid phase moving through the membrane. The membranes are typically stacked 5 to 15 layers deep in a comparatively small cartridge to generate a much smaller footprint than columns with similar outputs.

Preferably, the anion exchange material is an anion exchange membrane. Anion exchange membranes may be microporous or macroporous. In some embodiments, the anion exchange membrane is macroporous, optionally having a nominal pore size of at least about 1 μm, at least about 2 μm, or at least about 3 μm, or at least about 4 μm. In preferred embodiments, the anion exchange membrane has a nominal pore size of at least about 3 μm.

In preferred embodiments, the anion exchange membrane comprises quaternary ammonium (Q) groups, preferably with a nominal pore size of at least about 3 μm. Anion exchange membrane chromatography products such as those produced by Pall (e.g. Mustang® series) and Sartorius (e.g. Sartobind® series) may be suitable for use in the methods of the present invention. In some embodiments, the anion exchange step of the methods of the present invention comprise using a Sartobind® Q (Sartorius) or Mustang® Q (Pall Corporation) anion exchange membrane. In particular, the present inventors recognised that a Sartobind® Q membrane (e.g. Sartobind® Q, e.g. Sartobind® Q 4 mm or 8 mm) may provide improved scalability over alternative anion exchange membranes (e.g. Mustang® Q).

Other anion membrane absorbers and resins that may be suitable for use in the methods of the invention include but are not limited to Source 15Q and Source 30Q (Cytiva), Q-Sepharose XL (Cytiva), Fractogel® TMAE (Millipore), Adsept Q™ (Natrix Separations), and CIM® QA (BIA separations) Natrix Q (EMD Millipore), POROS XQ (ThermoFisher), Nuvia Q (BioRad), MacroPrep HighQ (BioRad), GigaCapQ 650M (Tosoh) and Capto Q (Cytiva).

The clarified sample (e.g. clarified lysate) is loaded onto the anion exchange chromatography material, e.g. anion exchange membrane. Surprisingly, the inventors found that the volumetric load could be reduced compared to prior art adenovirus purification methods. Accordingly, in embodiments in which the anion exchange chromatography material is a membrane (e.g. Sartobind® Q membrane), the clarified sample (e.g. clarified lysate) may be loaded at a load of up to about 70 L clarified sample/L anion exchange membrane, up to about 65 L clarified sample/L anion exchange membrane, up to about 60 L clarified sample/L anion exchange membrane, up to about 55 L clarified sample/L anion exchange membrane, up to about 50 L clarified sample/L anion exchange membrane, up to about 45 L clarified sample/L anion exchange membrane or up to about 40 L clarified sample/L anion exchange membrane. In preferred embodiments, the clarified sample (e.g. clarified lysate) is loaded at a load of about 50 L clarified sample/L anion exchange membrane.

In some embodiments in which the anion exchange chromatography material is a membrane (e.g. Sartobind® Q membrane), the clarified sample (e.g. clarified lysate) may be loaded at a load of between about 10 and about 75 L clarified sample/L anion exchange membrane, between about 20 and about 70 L clarified sample/L anion exchange membrane, or between about 50 and about 60 clarified sample/L anion exchange membrane.

In some embodiments, the clarified sample (e.g. clarified lysate) is set to have a flow rate of about 10 membrane volumes/min or less, preferably about 7 membrane volumes/min or less, most preferably about 5.5 membrane volumes/min or less. In preferred embodiments, the clarified sample (e.g. clarified lysate) is set to have a flow rate of about 5 membrane volumes/min.

Prior to loading, the clarified sample (e.g. clarified lysate) may be adjusted to increase the conductivity in the load, e.g. with NaCl, such as 5 M NaCl. Increasing the conductivity in the load may reduce binding of process-related impurities such as HCPs to the anion exchange material and improve binding capacity. In some embodiments, the adjusted clarified sample (e.g. adjusted clarified lysate) has a conductivity at 25° C. of from about 15 to about 30 mS/cm, such as from about 15 to about 26 mS/cm, preferably from about 15 to about 25 mS/cm. In some embodiments, the clarified sample (e.g. clarified lysate) is adjusted with from about 0.020 to about 0.040 kg 5 M NaCl/kg clarified sample, preferably from about 0.027 to about 0.033 kg 5 M NaCl/kg clarified sample, most preferably about 0.030 kg 5 M NaCl/kg clarified sample.

Optionally, the anion exchange step may comprise a load filter step in which the clarified sample (e.g. clarified lysate) is subjected to microfiltration prior to loading onto the anion exchange chromatography membrane. Such microfiltration may assist in mitigating high pressure over the anion exchange membrane. The particular microfiltration membrane selected will have pores of a size sufficiently large for adenovirus to pass through but small enough to effectively clear impurities. In some embodiments, the microfiltration membrane has a pore size of less than about 1 μm, less than about 0.75 μm, less than about 0.5 μm, or about less than about 0.25 μm. In preferred embodiments, the microfiltration membrane pore size is about 0.2 μm. Suitable membrane materials for microfiltration may include regenerated cellulose, polyethersulfone, polysulfone, or derivatives thereof, preferably polyethersulfone. In preferred embodiments, the microfiltration membrane comprises polyethersulfone and has a pore size of about 0.2 μm. A suitable microfiltration membrane for use in the methods of the present invention is the Millipore Express® SHC 0.5/0.2 μm filter.

After loading the clarified sample (e.g. clarified lysate), the anion exchange material may be washed with one or more buffers. In some embodiments, the anion exchange material is washed with a combination of buffers, e.g. an equilibration buffer followed by a wash buffer.

It will be understood that the pH of the equilibration and wash buffers will be high enough for adenovirus to bind (greater than approximately 6.5) and low enough to avoid viral instability. The precise maximum pH which is usable may depend on the specific stability profile of the adenovirus serotype and the buffer components. For example, the pH for a chimpanzee adenovirus may potentially range from about 6-10, preferably about 6.5-9, more preferably about 7.5-8.5, such as about 8.

The conductivity of the equilibration buffer at 25° C. may be in the range of about 1.5-3.5 mS/cm, about 2-3.2 mS/cm, or about 2.1-3.1 mS/cm. In preferred embodiments, the conductivity of the equilibration buffer at 25° C. is in the range of about 2.1-3.1 mS/cm. Thus, in preferred embodiments, the equilibration buffer has a pH of about 8 and a conductivity at 25° C. in the range of about 2.1-3.1 mS/cm. In particularly preferred embodiments, the equilibration buffer comprises about 50 mM tris, about 1 mM MgCl₂ and about 5% (w/v) sucrose, and has a pH of about 8.

The conductivity of the wash buffer at 25° C. may be in the range of about 15-30 mS/cm, about 18-27 mS/cm or about 20-24 mS/cm. In preferred embodiments, the conductivity of the wash buffer at 25° C. is in the range of about 20-24 mS/cm. The removal of unbound material from the anion exchange material may be assisted by the use of NaCl or KCl, preferably NaCl. Accordingly, in preferred embodiments, the wash buffer comprises NaCl at a concentration of up to about 100 mM, up to about 150 mM, up to about 200 mM, up to about 250 mM, or up to about 300 mM at pH 8. Most preferably, the wash buffer comprises NaCl at a concentration of up to about 222 mM, at pH 8. Thus, in preferred embodiments, the wash buffer comprises about 222 mM NaCl, has a pH of about 8, and a conductivity at 25° C. in the range of about 20-24 mS/cm. In particularly preferred embodiments, the wash buffer comprises about 50 mM tris, about 222 mM NaCl, about 1 mM MgCl₂ and about 5% (w/v) sucrose), and has a pH of about 8.

The bound product may be eluted with an elution buffer. The conductivity of the elution buffer at 25° C. may be in the range of about 25-50 mS/cm, about 30-45 mS/cm or about 35-43 mS/cm. In preferred embodiments, the conductivity of the elution buffer at 25° C. is in the range of about 35-43 mS/cm. The elution buffer may comprise NaCl at a concentration of up to about 300 mM, up to about 350 mM, up to about 400 mM, up to about 450 mM, or up to about 500 mM at pH 8. Most preferably, the elution buffer comprises NaCl at a concentration of up to about 444 mM, at pH 8. Thus, in preferred embodiments, the elution buffer comprises about 444 mM NaCl, has a pH of about 8, and a conductivity at 25° C. in the range of about 35-43 MS/CM. In particularly preferred embodiments, the elution buffer comprises about 50 mM tris, about 444 mM NaCl, about 1 mM MgCl₂ and about 5% (w/v) sucrose, and has a pH of about 8.

The elution buffer may be set to have a flow rate of less than about 10 membrane volumes/min, preferably about 7 membrane volumes/min or less, most preferably about 5.5 membrane volumes/min or less. In preferred embodiments, the elution buffer is set to have a flow rate of about 5 membrane volumes/min.

Following elution, the eluted product may be diluted with dilution buffer. In preferred embodiments, the dilution buffer comprises about 35 mM NaCl, about 10 mM histidine/histidine-HCl, about 1 mM MgCl₂, about 0.1 mM EDTA, about 7.5% (w/v) sucrose and about 0.5% (v/v) ethanol, and has a pH of about 6.6. The dilution buffer may be added to the eluted product at a 1:1 dilution ratio.

It will be understood that the volume of elution buffer directly affects the volume of the eluted product, and therefore the product from the anion exchange step. In the methods of the invention, a volume of elution buffer of from about 4.5 to about 5.5 membrane volumes, preferably about 5 membrane volumes may be used. In some embodiments, a dilution buffer volume of from about 3 membrane volumes to about 7 membrane volumes, preferably from about 3.5 membrane volumes to about 5.5 membrane volumes, preferably about 5 membrane volumes is used. In preferred embodiments, an elution buffer volume of 5 membrane volumes and a dilution buffer volume of 5 membrane volumes is used. By using such volumes, the methods can provide in a reduced anion exchange product volume and a more concentrated process stream. This enables a reduction in the membrane surface area that is required for the subsequent TFF processing step, thereby reducing the consumption of raw materials, eliminating large volume waste stream and shortening processing time.

The resulting material from the anion exchange chromatography step is anion exchange product.

The anion exchange product may have a pH of from 7 to about 9, preferably from about 7.5 to about 8.5. In some embodiments, the anion exchange product has a conductivity at 25° C. of from about 5 mS/cm to about 35 mS/cm, from about 10 mS/cm to about 30 mS/cm, preferably from about 15 mS/cm to about 25 mS/cm. Accordingly, in some embodiments, the anion exchange product has a pH of from about 7.5 to about 8.5 and a conductivity at 25° C. of from about 15 mS/cm to about 25 mS/cm.

The anion exchange product may optionally undergo microfiltration for microbial control. The particular microfiltration membrane selected will have pores of a size sufficiently large for adenovirus to pass through but small enough to effectively clear impurities. In some embodiments, the microfiltration membrane has a pore size of less than about 1 μm, less than about 0.75 μm, less than about 0.5 μm, or about less than about 0.25 μm. In preferred embodiments, the microfiltration membrane pore size is about 0.2 μm. Suitable membrane materials for microfiltration may include regenerated cellulose, polyethersulfone, polysulfone, or derivatives thereof, preferably polyethersulfone. In preferred embodiments, the microfiltration membrane comprises polyethersulfone and has a pore size of about 0.2 μm. A suitable microfiltration membrane for use in the methods of the present invention is the Millipore Express® SHC 0.5/0.2 μm filter.

In a preferred step, the methods of the present invention comprise a further processing step following anion exchange by mixed mode size exclusion chromatography to provide a mixed mode size exclusion product. The mixed mode size exclusion chromatography can be performed with mixed mode size exclusion resins including but not limited to Capto Core 700 (Cytiva), Capto Core 400 (Cytiva) and Monomix Core 60 (Sepax Technologies)

### Tangential Flow Filtration

Following anion exchange or mixed mode size exclusion chromatography, the methods of the present invention comprise a tangential flow filtration (TFF) step. The TFF step comprises ultrafiltration and diafiltration to concentrate the anion exchange product and to introduce a buffer, respectively. Thus, in the methods of the present invention, the TFF product is reduced in volume and has higher adenovirus concentration compared to load for the TFF step.

The optimum feed flow may be determined by a person skilled in the art. In preferred embodiments, the feed flow is set to from about 1 to about 15 litres/m²/min (LMM), preferably from about 2 to about 10 LMM, most preferably from about 3 to about 7 LMM, such as 5 LMM.

The load for the TFF step is the anion exchange product or mixed mode size exclusion product. Optionally, the anion exchange product is processed by depth filtration prior to TFF. Such a depth filtration step may provide benefit in terms of HCP reduction and improved membrane lifetime. Suitable depth filters include those discussed in the above Clarification section. In preferred embodiments, a depth filter having a nominal filter rating of greater than 0 μm and up to about 0.1 μm is used, e.g. a Millistak+® Pod depth filter, XOHC media or a Millistak+® HC Pro Pod depth filter, XOSP media. In particularly preferred embodiments, a Millistak+® Pod XOHC depth filter is used.

Depending on the manufacturer and membrane type, nominal molecular weight cutoffs (NMWCO) for the ultrafiltration membrane may be between 100 and 1000 kDa. The membrane composition may be, but is not limited to, regenerated cellulose, polyethersulfone, polysulfone, or derivatives thereof. These membranes can be flat sheets or hollow fibres. Turbulence-promoting screens may also be useful to optimize impurity clearance. In some embodiments, 300 kDa or 500 kDa NMWCO polyethersulfone flat sheet membranes with a turbulence-promoting screen (e.g. Pellicon® 2 membrane, 300 kDa MWCO, C screen) are used. The tangential flow filtration may be controlled by setting both the cross-flow and the permeate flux and maintaining the transmembrane pressure at and or below a fixed pressure limit.

The membrane volumetric load may be determined by a person skilled in the art. In preferred embodiments, the membrane volumetric load is less than about 100 L/m².

Ultrafiltration may concentrate the anion exchange product or mixed mode size exclusion product. For example, in some embodiments of the methods of the invention, ultrafiltration concentrates the anion exchange product or mixed mode size exclusion product to a volume that is from about 0.03 to about 0.3 L/L nuclease-treated lysate, preferably about 0.05 L/L nuclease-treated lysate. In some embodiments, during the concentration step the permeate flow is set to less than about 1.00 LMM, preferably less than about 0.90 LMM, most preferably less than about 0.80 LMM. In particularly preferred embodiments, the permeate flow is set to about 0.67 LMM during the concentration step.

The concentrated anion exchange or mixed mode size exclusion product is then diafiltered with diafiltration buffer. Diafiltration may be operated using at least 9, at least 10, at least 11, or at least 12 diavolumes of diafiltration buffer. In preferred embodiments, diafiltration is operated using at least 10 diavolumes of diafiltration buffer. A permeate flow of less than 0.80LMM, such as 0.67 LMM, may be used. The pH of the diafiltration buffer may be in the range of about 6-8, preferably about 6-7, more preferably about 6.5-6.7. The conductivity of the diafiltration buffer at 25° C. may be in the range of about 1-8 mS/cm, preferably about 2-6 mS/cm, more preferably about 3-4.6 mS/cm. Thus, in preferred embodiments, the diafiltration buffer has a pH of about 6.5-6.7 and a conductivity at 25° C. in the range of about 3-4.6 mS/cm. In some embodiments, the diafiltration buffer does not comprise Polysorbate-80. In preferred embodiments, the diafiltration buffer comprises about 35 mM NaCl, about 10 mM histidine/histidine-HCl, about 1 mM MgCl₂, about 0.1 mM EDTA, about 7.5% (w/v) sucrose and about 0.5% (v/v) ethanol, and has a pH of about 6.6.

The diafiltered product is recovered from the TFF system. The diafiltered product may be recovered by flushing with a volume of diafiltration buffer (e.g. at least 1, at least 2, at least 3, at least 4, or at least 5 system hold up volumes of diafiltration buffer). In preferred embodiments, the diafiltered product is recovered by flushing with 3 system hold up volumes of diafiltration buffer. Preferably, the same diafiltration buffer is used as for the diafiltration step.

The resulting material from the TFF step is TFF product. The TFF product may have one or more, preferably all, of the properties set forth in Table 1.

TABLE 1
Properties of TFF product

| Property | Specification |
|---|---|
| pH | 6.1-7.1 |
| Conductivity (mS/cm at 25° C.) | ≤5.0 |
| Infectivity (ifu/mL) | ≥2.4 × 10⁸ |
| Host Cell DNA (ng/0.5 × 10¹¹ vp) | ≤10 |
| Host Cell Protein (ng/0.5 × 10¹¹ vp) | ≤200 |

vp-adenovirus particles

### Formulation

The TFF product may be formulated to provide a formulated product. In some embodiments, the formulation step comprises adding Polysorbate-80 to the TFF product. In some embodiments, Polysorbate-80 is added to achieve a concentration of about 0.1% (w/v) Polysorbate-80 in the formulated product. In preferred embodiments, the formulated product has the following composition: about 35 mM NaCl, about 10 mM histidine/histidine-HCl, about 1 mM MgCl₂, about 0.1 mM EDTA, about 7.5% (w/v) sucrose, about 0.1% (w/v) Polysorbate-80 and about 0.5% (v/v) ethanol, and has a pH of about 6.6.

### Sterile Filtration

The formulated product may undergo sterile filtration. The sterile filtration step may be conducted using a filter having a pore size of a size that is sufficiently small to retain microbes and sufficiently small to allow passage of adenovirus. In preferred embodiments, the filter has a pore size of about 0.2 μm. The filter may be constructed of a material that is well known in the art, such as polyethersulfone, PVDF, polypropylene, cellulose, cellulose esters, nylon or any other material which is consistent with low product binding. In preferred embodiments, the filter comprises hydrophilic polyethersulfone. In particularly preferred embodiments, the filter comprises a hydrophilic polyethersulfone membrane with a pore size of 0.2 μm (e.g. Pall Supor® EKV, 0.2 μm).

The product collected from the filter is the drug substance. The drug substance may be formulated in formulation buffer. In preferred embodiments, the formulation buffer comprises about 35 mM NaCl, about 10 mM histidine/histidine-HCl, about 1 mM MgCl₂, about 0.1 mM EDTA, about 7.5% (w/v) sucrose, about 0.1% (w/v) Polysorbate-80 and about 0.5% (v/v) ethanol, and has a pH of about 6.6. This may be achieved by equilibrating the filter with formulation buffer prior to filtration and by chasing with formulation buffer following filtration.

### Drug Substance

The methods of the invention may result in the production of a drug substance.

The methods of the invention may provide a drug substance with an increased concentration of adenovirus (e.g. at least about 10-fold, at least about 20-fold, at least about 30-fold greater adenovirus concentration) compared with alternative adenovirus purification methods (e.g., the method of Example 1). An advantage of having a more concentrated drug substance is that it does not require as much storage space, which may be particularly advantageous when stored at −80° C. In some embodiments, the drug substance has an adenovirus virus particle (also referred to herein as “virus particle”) concentration of at least about 0.8×10¹¹ vp/mL, at least about 1×10¹¹ vp/mL, at least about 1.2×10¹¹ vp/mL, at least about 1.4×10¹¹ vp/mL, at least about 1.6×10¹¹ vp/mL, at least about 1.8×10¹¹ vp/mL, at least about 2×10¹¹ vp/mL, at least about 2.2×10¹¹ vp/mL, at least about 2.4×10¹¹ vp/mL, or at least about 2.6×10¹¹ vp/mL.

In some embodiments, the drug substance has one or more, preferably all, of the following properties: reduced HCP levels compared to the HCP levels in the nuclease-treated lysate; a pH of between about 6.1-7.1; an osmolality of greater than about 265 mOsm/Kg; an infectivity of greater than or equal to about 2.4×10⁹ ifu/mL; a virus particle concentration of greater than about 0.8×10¹¹ vp/mL; a DNA:protein ratio of about 1.1-1.6; a virus particle:infectious titer ratio of less than or equal to about 500:1 vp/ifu; less than about 10 ng/dose host cell DNA; less than about 200 ng/dose HOP; less than about 20 ng/mL Benzonase®; less than about 10 EU/mL endotoxin; less than about 5 CFU/10 mL bioburden. As used herein, a dose of drug substance comprises about 5×10¹⁰ virus particles.

In some embodiments, the drug substance has one or more, preferably all, of the properties set forth in Table 2. For example, in some embodiments, the drug substance has an infectivity of greater than or equal to about 2.4×10⁹ ifu/mL and/or a virus particle concentration of at least about 0.8×10¹¹ vp/mL, such as a virus particle concentration of at least about 2×10¹¹ vp/mL. In preferred embodiments, the drug substance has an infectivity of greater than or equal to about 2.4×10⁹ ifu/mL and a virus particle concentration of at least about 0.8×10¹¹ vp/mL, such as a virus particle concentration of at least about 2×10¹¹ vp/mL.

The methods of the invention result in a drug substance containing low levels of HCPs. In some embodiments, the drug substance comprises HCPs at a concentration of 2000 ng or less per dose, such as 1000 ng or less per dose, 500 ng or less per dose, 200 ng or less per dose, 100 ng or less per dose, or 50 ng or less per dose. In preferred embodiments, the drug substance comprises HCPs at a concentration of 1000 ng or less per dose. In some embodiments, the drug substance comprises host cell DNA at a concentration of 100 ng or less per dose, such as 50 ng or less per dose, 25 ng or less per dose, 10 ng or less per dose or 5 ng or less per dose. In preferred embodiments, the drug substance comprises host cell DNA at a concentration of 10 ng or less per dose.

TABLE 2
Properties of drug substance.

| Property | Specification |
|---|---|
| pH | 6.1-7.1 |
| Osmolality (mOsm/kg) | >265 |
| Infectivity (ifu/mL) | ≥2.4 × 10⁹ |
| Virus Particle Concentration (vp/mL) | >0.8 × 10¹¹ |
| DNA: Protein Ratio (A260/A280) | 1.1-1.6 |
| Virus Particle: Infectious Titer Ratio (vp/ifu) | ≤500:1 |
| Host Cell DNA (ng/dose) | <10 |
| Host Cell Protein (ng/dose) | <200 |
| Benzonase (ng/mL) | <20 |
| Endotoxin (EU/mL) | <10 |
| Bioburden (CFU/10 mL) | <5 |
| Polysorbate-80 (%) | <0.15 |

The drug substance may be stored at between about −90 and −55° C., optionally with a 2-8° C. hold of up to 5 days prior to freeze.

In some embodiments, the drug substance is sterile filtered and diluted about 20× to form a drug product. Advantageously, the drug product may be stored at 2-8° C. long-term, for example for at least about 1 week, at least about 2 weeks, at least about 1 month, or at least about 1 year.

### Adenovirus Vector

In preferred embodiments of the methods of the invention, the adenovirus is an adenovirus vector. As used herein “adenovirus vector” means a form of an adenovirus which has been modified for insertion of a nucleotide sequence encoding a heterologous gene into a eukaryotic cell. As used herein, “heterologous gene” means a gene derived from a genotypically distinct entity from that of the rest of the entity to which it is being compared. Thus, a heterologous gene refers to any gene that is not isolated from, derived from, or based upon a naturally occurring gene of the adenovirus. As used herein “naturally occurring” means found in nature and not synthetically prepared or modified.

In preferred embodiments of the methods of the invention, the adenovirus vector comprises a heterologous gene encoding a protein of interest, for example a therapeutic protein or an immunogenic protein. Alternatively, a heterologous gene may include a reporter gene, which upon expression produces a detectable signal. Such reporter genes include, without limitation, DNA sequences encoding β-lactamase, β-galactosidase (LacZ), alkaline phosphatase, thymidine kinase, green fluorescent protein (GFP), chloramphenicol acetyltransferase (CAT), luciferase, membrane bound proteins including, for example, CD2, CD4, CD8, the influenza hemagglutinin protein, and others well known in the art, to which high affinity antibodies directed thereto exist or can be produced by conventional means, and fusion proteins comprising a membrane bound protein appropriately fused to an antigen tag domain from, among others, hemagglutinin or Myc. These coding sequences, when associated with regulatory elements which drive their expression, provide signals detectable by conventional means, including enzymatic, radiographic, colorimetric, fluorescence or other spectrographic assays, fluorescent activating cell sorting assays and immunological assays, including enzyme linked immunosorbent assay (ELISA), radioimmunoassay (RIA) and immunohistochemistry.

In preferred embodiments, the heterologous gene is a sequence encoding a product, such as protein, RNA, enzyme or catalytic RNA which is useful in biology and medicine, such as a therapeutic gene or an immunogenic gene. The heterologous gene may be used for treatment, e.g. of genetic deficiencies, as a cancer therapeutic, as a vaccine, for induction of an immune response, and/or for prophylactic purposes.

In preferred embodiments, the heterologous gene encodes a foreign antigen such as a naturally occurring form of a foreign antigen, or a modified form thereof. As used herein, “foreign antigen” means an antigen which induces a host immune response and is derived from a genotypically distinct entity from that of the host in which it induces the immune response. As used herein, a modified form of a foreign antigen means a form of the foreign antigen which induces a host immune response against the naturally occurring antigen and has at least 50%, at least 60%, at least 70%, at least 80%, at least 85%, at least 90%, at least 95%, at least 98%, or at least 99% sequence identity to the naturally occurring antigen. As used herein, induction of an immune response refers to the ability of a protein to induce a T cell and/or a humoral immune response to the protein. Determination of a host immune response against a naturally occurring form of a foreign antigen, or a modified form thereof, may be assessed by any suitable method such as those described in Jeyanathan et al. 2020; Immunological considerations for COVID-19 vaccine strategies; Nature Reviews Immunology 20, 615-632 and Albert-Vega et al. 2018; Immune Functional Assays, From Custom to Standardized Tests for Precision Medicine; Frontiers in Immunology 9:2367. In some embodiments, the modified form of the naturally occurring antigen induces a more powerful host immune response than that induced by the naturally occurring antigen. In some embodiments, the modified form of the naturally occurring antigen induces a weaker host immune response than that induced by the naturally occurring antigen.

In some embodiments, the foreign antigen is derived from SARS-CoV2, preferably from the spike protein of SARS-CoV2. SARS-CoV2 is a newly-emergent coronavirus which causes a severe acute respiratory disease, COVID-19. Thus far, no vaccine has been available on a global scale to prevent SARS-CoV2 infection. Because this virus uses its spike glycoprotein for interaction with the cellular receptor ACE2 and the serine protease TMPRSS2 for entry into a target cell, this spike protein represents an attractive target for vaccine therapeutics. Accordingly, in preferred embodiments, the heterologous gene codes for a naturally occurring form of the SARS-CoV2 spike protein, or a modified version thereof. The RNA, DNA, and amino acid sequence of the SARS-CoV2 spike protein are known to those skilled in the art and can be found in many databases, for example, in the database of the National Center for Biotechnology Information (NCBI), where it has an accession number of NC_045512.2. For example, in some embodiments, the heterologous gene encodes the SARS-CoV2 spike protein comprising an amino acid sequence set forth in SEQ ID NO: 1. In other exemplary embodiments, the heterologous gene encodes a modified form of the SARS-CoV2 spike protein comprising an amino acid sequence set forth in SEQ ID NO: 2. As will be readily understood, the amino acid sequence set forth in SEQ ID NO: 2 comprises the SARS-CoV2 spike protein amino acid sequence with the signal peptide of the human tissue plasminogen activator gene (tPA) at the N terminus. Presence of the N-terminal tPA sequence may enhance immunogenicity of the SARS-CoV2 spike protein.

In addition to the heterologous gene, the vector may also include conventional control elements which are operably linked to the heterologous gene in a manner that permits its transcription, translation and/or expression in a cell infected with the adenovirus. As used herein “operably linked” includes both expression control sequences that are contiguous with the gene of interest and expression control sequences that act in trans or at a distance to control the gene of interest.

Expression control sequences may include appropriate transcription initiation, termination, promoter and enhancer sequences; efficient RNA processing signals such as splicing and polyadenylation (poly A) signals; sequences that enhance translation efficiency; sequences that enhance protein stability; and when desired, sequences that enhance secretion of the encoded product. As used herein, a “promoter” is a nucleotide sequence that permits binding of RNA polymerase and directs the transcription of a gene. A number of expression control sequences, including promoters which are internal, native, constitutive, inducible and/or tissue-specific are known in the art and may be utilized.

The adenovirus vector may be derived from a mammalian adenovirus. In some embodiments of the method of the invention, the adenovirus vector is derived from a human adenovirus. In some embodiments, the human adenovirus is a serotype 5 human adenovirus. In some embodiments, the human adenovirus is not a serotype 5 human adenovirus.

In preferred embodiments, the adenovirus vector is not derived from a human adenovirus. Thus, the adenovirus vector may be derived from a non-human adenovirus, for example, a chimpanzee adenovirus. In particularly preferred embodiments, the adenovirus vector is derived from a chimpanzee adenovirus, e.g. ChAdOx1 (Antrobus et al. 2014 Mol. Ther. 22(3):668-674), ChAdOx2 (Morris et al. 2016 Future Virol. 11(9):649-659), ChAd3 or ChAd63. In especially preferred embodiments, the adenovirus vector is derived from ChAdOx1.

In some embodiments of the methods of the invention, the adenovirus vector is for use in a vaccine and is derived from the same species as the species for which the vaccine is targeted. For example, in some embodiments, the vaccine is targeted to a disease found in humans and the adenovirus vector is derived from a human adenovirus. In preferred embodiments, however, the adenovirus vector is for use in a vaccine and is derived from a species different from that for which the vaccine is targeted. For example, in some embodiments, the vaccine is targeted to a disease found in humans and the adenovirus vector is derived from a non-human adenovirus, such as a chimpanzee adenovirus. It is thought that the use of an adenovirus vector derived from a species different from the species for which a vaccine is targeted may provide an improved vaccine that encounters a lower incidence of pre-existing anti-adenoviral immunity when administered.

Adenovirus vectors may be engineered so that they are unable to replicate after administration to a host. Accordingly, in some embodiments of the methods of the invention, the adenovirus vector is a replication deficient adenovirus vector (e.g. replication deficient adenovirus vector derived from chimpanzee adenovirus). As used herein, a “replication deficient adenovirus vector” means an adenovirus vector which is unable to replicate in a host cell lacking one or more adenovirus replication genes. In some embodiments, the adenovirus vector lacks an E1A gene. In some embodiments, the adenovirus vector has been modified to prevent elimination of cells infected with the adenovirus vector by the host immune system. For example, in some embodiments, the adenovirus vector lacks an E1B gene and/or an E3 gene. In some embodiments, the adenovirus vector lacks an E1B gene. In some embodiments, the adenovirus vector lacks an E3 gene. In some embodiments, the adenovirus vector lacks an E1B gene and an E3 gene. In some embodiments, the adenovirus vector is a minimal adenovirus vector comprising an origin of replication (ori) and a packaging sequence. In some embodiments, the minimal adenovirus vector further comprises a heterologous gene encoding a protein of interest.

### Host Cell Population

In preferred embodiments of the methods of the invention, the host cell population is complementary to the adenovirus added to the cell population. As used herein, a “host cell population complementary to an adenovirus being produced” is a host cell population which has been engineered to express an adenovirus factor which is not expressed by the adenovirus being produced. For example, in some embodiments, the adenovirus does not express an adenovirus DNA replication factor and the host cell population expresses the adenovirus DNA replication factor. As used herein an “adenovirus DNA replication factor” is a factor which in nature, forms part of the adenovirus DNA, and is required for the adenovirus to replicate in a host cell. Accordingly, in some embodiments, the adenovirus does not express an E1A protein, an E1B protein, and/or an E4 protein and the cell population expresses the E1A protein, the E1B protein, and/or the E4 protein.

The host cell population may comprise cells in suspension.

The host cell population may be a primary cell population which has been freshly isolated from a tissue. In some embodiments, the tissue is a mammalian tissue.

Alternatively, the host cell population may be derived from a cell line which has been adapted for culture. In some embodiments, the cell line is an immortalised cell line. In some embodiments, the cell line is a mammalian cell line. In some embodiments, the host cell population comprises mammalian cells. For example, in some embodiments the host cell population comprises human embryonic kidney (HEK) cells or is a HEK cell line. The mammalian cells may express an adenovirus replication factor. For example, in some embodiments, the host cell population expresses an E1A protein, an E1B protein, and/or an E4 protein. In some embodiments, the host cell population expresses a tetracycline repressor protein. In preferred embodiments, the host cell population comprises T-REx™ cells. In some embodiments, the host cell population consists of T-REx™ cells.

Unless defined otherwise, all technical and scientific terms used herein have the same meaning as commonly understood by one of ordinary skill in the art to which this disclosure belongs. Singleton, et al., DICTIONARY OF MICROBIOLOGY AND MOLECULAR BIOLOGY, 20 ED., John Wiley and Sons, New York (1994), and Hale & Marham, THE HARPER COLLINS DICTIONARY OF BIOLOGY, Harper Perennial, NY (1991) provide the skilled person with a general dictionary of many of the terms used in this disclosure.

This disclosure is not limited by the exemplary methods and materials disclosed herein, and any methods and materials similar or equivalent to those described herein can be used in the practice or testing of embodiments of this disclosure.

Unless otherwise indicated, any nucleic acid sequences are written left to right in 5′ to 3′ orientation; amino acid sequences are written left to right in amino to carboxy orientation, respectively.

The headings provided herein are not limitations of the various aspects or embodiments of this disclosure. In addition, it will be understood that any of the embodiments described herein are applicable to any of the aspects described herein.

Concentrations, amounts, volumes, percentages and other numerical values may be presented herein in a range format. Numeric ranges are inclusive of the numbers defining the range. Where a range of values is provided, it is understood that each intervening value, to the tenth of the unit of the lower limit unless the context clearly dictates otherwise, between the upper and lower limits of that range is also specifically disclosed. Each smaller range between any stated value or intervening value in a stated range and any other stated or intervening value in that stated range is encompassed within this disclosure. The upper and lower limits of these smaller ranges may independently be included or excluded in the range, and each range where either, neither or both limits are included in the smaller ranges is also encompassed within this disclosure, subject to any specifically excluded limit in the stated range. Where the stated range includes one or both of the limits, ranges excluding either or both of those included limits are also included in this disclosure. It is also to be understood that such range format is used merely for convenience and brevity and should be interpreted flexibly to include not only the numerical values explicitly recited as the limits of the range but also to include all the individual numerical values or sub-ranges encompassed within that range as if each numerical value and sub-range is explicitly recited.

It must be noted that as used herein and in the appended claims, the singular forms “a”, “an”, and “the” include plural referents unless the context clearly dictates otherwise. Thus, for example, reference to “an agent” includes a plurality of such agents and reference to “the agent” includes reference to one or more agents and equivalents thereof known to those skilled in the art, and so forth.

“About” may generally mean an acceptable degree of error for the quantity measured given the nature or precision of the measurements. Exemplary degrees of error are within 20 percent (%), typically, within 10%, and more typically, within 5% of a given value or range of values. Preferably, the term “about” shall be understood herein as plus or minus (±) 5%, preferably ±4%, ±3%, ±2%, ±1%, ±0.5%, ±0.1%, of the numerical value of the number with which it is being used.

Embodiments described herein as “comprising” one or more features may also be considered as disclosure of the corresponding embodiments “consisting essentially of” such features, or “consisting of” such features.

The above embodiments are to be understood as illustrative examples. Further embodiments are envisaged. It is to be understood that any feature described in relation to any one embodiment may be used alone, or in combination with other features described, and may also be used in combination with one or more features of any other of the embodiments, or any combination of any other of the embodiments. Furthermore, equivalents and modifications not described above may also be employed without departing from the scope of the invention, which is defined in the accompanying claims.

All documents cited herein are each entirely incorporated by reference herein, including all data, tables, figures, and text presented in the cited documents.

### EXAMPLES

### Example 1: Adenovirus Purification Using Process A

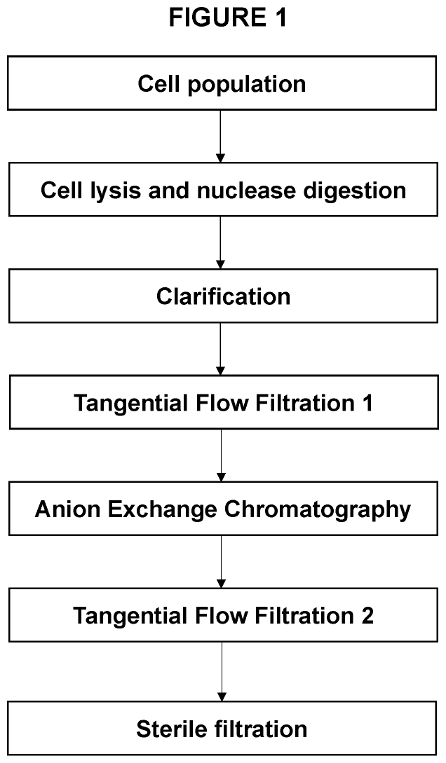

Adenovirus purification using Process A is depicted in FIG. 1. Further details are provided below.

### Cell Lysis and Benzonase® Digestion

10× lysis buffer (500 mM tris, 20 mM MgCl₂, 50% (w/v) sucrose, 10% (v/v) polysorbate (PS) 20, pH 8.0) was added to the bioreactor to a final concentration of 1× lysis buffer to lyse the cells within the culture. Within 30 minutes of cell lysis, Benzonase® stock solution was mixed with cell culture medium and added to the bioreactor to a final concentration of 15 Units/mL lysate. Cell lysis and Benzonase® treatment were continued for a minimum of two hours prior to the start of harvest.

The bioreactor temperature of 37° C. and agitation rate of 60 RPM were maintained from the cell culture stage of the bioreactor for the duration of this unit operation while pH control and dissolved oxygen were turned off.

### Clarification

The Benzonase®-treated lysate was harvested from the bioreactor and clarified using depth filtration.

The depth filters (Millistak+® HC Pro Pod depth filter, COSP media) were first flushed with water for injection and then equilibrated with equilibration buffer (50 mM tris, 2 mM MgCl₂, 5% (w/v) sucrose, 1% (v/v) PS 20, pH 8.0) prior to initial loading. Benzonase®-treated lysate was then loaded onto the depth filter. After loading, chase buffer (50 mM tris, 2 mM MgCl₂, 5% (w/v) sucrose, 1% (v/v) PS 20, pH 8.0) was added to the depth filter. Product collection began after 75% of filter hold up volume was diverted to waste in order to reduce product dilution and ended at the conclusion of chase.

The bioreactor agitation rate was maintained from the lysis and Benzonase® digestion steps for the duration of this unit operation while temperature control was turned off.

### Tangential Flow Filtration and Bioburden Removal Filtration 1

Prior to anion exchange chromatography, the clarified lysate was subjected to tangential flow filtration followed by 0.2 μm filtration to remove impurities. The clarified lysate was first concentrated by ultrafiltration using a 300 kDa ultrafiltration membrane (Pellicon 2 PES membrane, 300 kDa MWCO, C Screen). The concentrated product was then diafiltered with diafiltration buffer (59 mM bis-tris, 100 mM NaCl, 1 mM MgCl₂, 5% (w/v) sucrose, 0.1% (v/v) PS 20, pH 7.0) before being subjected to a further concentration step and diafiltration step with diafiltration buffer.

The tangential flow filtration 1 product was equilibrated with equilibration buffer (59 mM bis-tris, 100 mM NaCl, 1 mM MgCl₂, 5% (w/v) sucrose, 0.1% (v/v) PS 20, pH 7.0). The equilibrated product was then filtered (using Pall Supor EKV, 0.2 μm) and chased using chase buffer (59 mM bis-tris, 100 mM NaCl, 1 mM MgCl₂, 5% (w/v) sucrose, 0.1% (v/v) PS 20, pH 7.0) into an intermediate holding vessel.

### Anion Exchange Chromatography

The anion exchange chromatography step is designed to bind the product and remove process-related impurities. Anion exchange chromatography took place using the Mustang® Q system. Accordingly, the anion exchange chromatography membrane (Mustang Q®) was first flushed with 1 M NaCl before being sanitized with 20 membrane volumes of sanitization buffer (1 N NaOH) and then conditioned with conditioning buffer (1 M NaOH).

The anion exchange chromatography membrane was pre-equilibrated with pre-equilibration buffer (50 mM bis-tris, 100 mM NaCl, 1 mM MgCl₂, 5% (w/v) sucrose, 0.1% (v/v) PS 20, pH 7.0) before being activated with 30 membrane volumes of activation buffer (59 mM bis-tris, 444 mM NaCl, 1 mM MgCl₂, 5% (w/v) sucrose, pH 7.0) and equilibrated with 40 membrane volumes of equilibration buffer (59 mM bis-tris, 100 mM NaCl, 1 mM MgCl₂, 5% (w/v) sucrose, 0.1% (v/v) PS 20, pH 7.0) prior to initiating load.

The filtered product from the tangential flow filtration and bioburden removal filtration 1 step was loaded onto the anion exchange chromatography membrane. After loading, the anion exchange chromatography membrane was washed with 40 membrane volumes of wash buffer (59 mM bis-tris, 222 mM NaCl, 1 mM MgCl₂, 5% (w/v) sucrose, pH 7.0). Product was eluted from the anion exchange chromatography membrane with 25 membrane volumes of elution buffer (59 mM bis-tris, 444 mM NaCl, 1 mM MgCl₂, 5% (w/v) sucrose, pH 7.0) and immediately diluted with AEX dilution buffer (35 mM NaCl, 10 mM histidine, 1 mM MgCl₂, 0.1 mM EDTA, 7.5% (w/v) sucrose, 0.1% (v/v) PS 80, 0.5% (v/v) ethanol, pH 6.6) to about half the initial cell lysate volume (equivalent to about 25 membrane volumes or more).

### Tangential Flow Filtration and Bioburden Removal Filtration 2

The anion exchange product was concentrated by ultrafiltration using a 300 kDa ultrafiltration membrane (Omega PES membrane, 300 kDa). The concentrated product was then diafiltered with diafiltration buffer (35 mM NaCl, 10 mM histidine, 1 mM MgCl₂, 0.1 mM EDTA, 7.5% (w/v) sucrose, 0.1% (v/v) PS 80, 0.5% (v/v) ethanol, pH 6.6). The diafiltered product is recovered from the TFF system by flushing with diafiltration buffer.

The bioburden reduction filter (Pall Supor EKV, 0.2 μm) was equilibrated with equilibration buffer (35 mM NaCl, 10 mM histidine, 1 mM MgCl₂, 0.1 mM EDTA, 7.5% (w/v) sucrose, 0.1% (w/v) PS 80, 0.5% (v/v) ethanol, pH 6.6). The diafiltered product was then filtered (Pall Supor EKV, 0.2 μm) and chased using chase buffer (35 mM NaCl, 10 mM histidine, 1 mM MgCl₂, 0.1 mM EDTA, 7.5% (w/v) sucrose, 0.1% (w/v) PS 80, 0.5% (v/v) ethanol, pH 6.6) into an intermediate holding vessel, after which it may be stored for up to 7 days at 2-8° C. prior to freeze at ≤−65° C.

### Example 2: Adenovirus Purification Using Process B

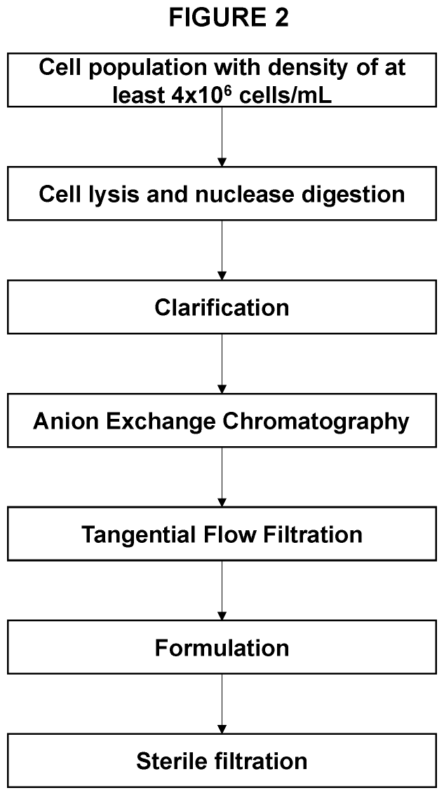

Adenovirus purification using Process B is depicted in FIG. 2. Further details are provided below.

### Cell Lysis and Benzonase® Digestion

The starting material was T-REx cells at a cell density of at least 4×10⁶ cells/mL. Lysis buffer (500 mM tris, 20 mM MgCl₂, 50% (w/v) sucrose, 10% (v/v) polysorbate (PS) 20, pH 8.0) was added to the bioreactor to a final concentration of 1× lysis buffer to lyse the cells within the culture. Within 30 minutes of cell lysis, Benzonase® stock solution was mixed with cell culture medium and added to the bioreactor to achieve a final concentration of 15,000 Units/kg lysate. Cell lysis and Benzonase® treatment were continued for a minimum of two hours prior to the start of harvest.

The bioreactor temperature of 33° C., agitation rate of 15-70 W/m³, and overlay were maintained from the cell culture stage of the bioreactor for the duration of this unit operation while pH control and dissolved oxygen were turned off.

### Clarification

The Benzonase®-treated lysate was harvested from the bioreactor and clarified using depth filtration, followed in series by 0.2 μm filtration. The depth filters (Millistak+® HC Pro Pod depth filter, COSP media) were first flushed with water for injection and then equilibrated with equilibration buffer (50 mM tris, 2 mM MgCl₂, 5% (w/v) sucrose, 1% (v/v) PS 20, pH 8.0) prior to initial loading. Following depth filtration, the Benzonase®-treated lysate was loaded onto the 0.2 μm filtration membrane (Millipore Express® SHC 0.2 μm filter). After loading, chase buffer (50 mM tris, 2 mM MgCl₂, 5% (w/v) sucrose, 1% (v/v) PS 20, pH 8.0) was added to the 0.2 μm filtration membrane. Product collection began after 75% of filter hold up volume was diverted to waste in order to reduce product dilution and ended at the conclusion of chase.

The bioreactor agitation rate and overlay were maintained from the lysis and Benzonase® digestion steps for the duration of this unit operation while pH control and dissolved oxygen were turned off. The temperature control setpoint was reduced to 20° C. prior to initiating clarification, but the unit operation was performed with load material at 15-35° C.

### Anion Exchange Chromatography

The anion exchange chromatography step is designed to bind the product and remove process-related impurities. The Sartobind® Q anion exchange chromatography system was used for this step. The anion exchange chromatography membrane (Sartobind Q®, 8 mm) was first sanitized with 30 membrane volumes of sanitization buffer (1 N NaOH or 0.5 N NaOH) and then activated with 10 membrane volumes of activation buffer (1 M NaOH).

Next, the anion exchange chromatography membrane was equilibrated with 20 membrane volumes of equilibration buffer (50 mM tris, 1 mM MgCl₂, 5% (w/v) sucrose) prior to initiating load. The clarified lysate was adjusted with 5 M NaCl and then loaded onto the membrane at 50 L clarified lysate/L membrane. After loading, the membrane was washed first with 10 membrane volumes of equilibration buffer (50 mM tris, 1 mM MgCl₂, 5% (w/v) sucrose), followed by 30 membrane volumes of wash buffer (50 mM tris, 222 mM NaCl, 1 mM MgCl₂, 5% (w/v) sucrose), pH 8.0).

Product was eluted from the anion exchange chromatography membrane with 5 membrane volumes of elution buffer (50 mM tris, 444 mM NaCl, 1 mM MgCl₂, 5% (w/v) sucrose, pH 8.0) and immediately diluted with 5 membrane volumes of AEX dilution buffer (35 mM NaCl, 10 mM histidine/histidine-HCl, 1 mM MgCl₂, 0.1 mM EDTA, 7.5% (w/v) sucrose, 0.5% (v/v) ethanol, pH 6.6) at a 1:1 dilution ratio. By using these volumes of elution and dilution buffer, the anion exchange product volume is reduced by a factor of about 5 or greater as compared to Process A. The decreased anion exchange product volume enables a reduction in the membrane surface area that is required for the subsequent TFF processing step, thereby reducing the consumption of raw materials, eliminating large volume waste stream and shortening processing time.

Following product collection, the membrane was flushed with the activation and sanitization buffers.

### Tangential Flow Filtration

The anion exchange product was concentrated by ultrafiltration using an ultrafiltration membrane (Pellicon 2 PES membrane, 300 kDa MWCO, C Screen) to a final volume that was 0.03-0.3 L/L nuclease-treated lysate. The concentrated product was then diafiltered with 10 diavolumes of diafiltration buffer (35 mM NaCl, 10 mM histidine/histidine-HCl, 1 mM MgCl₂, 0.1 mM EDTA, 7.5% (w/v) sucrose, 0.5% (v/v) ethanol, pH 6.6). The diafiltered product was recovered from the TFF system by flushing with three system hold-up volumes of diafiltration buffer.

### Formulation

The TFF product was formulated by the addition of 10% (w/v) PS-80 to reach a final PS 80 concentration of 0.1% (w/v).

### 0.2 Micron Bulk Filtration

The final 0.2 μm Drug Substance filter (Pall Supor EKV, 0.2 μm) was equilibrated with formulation buffer (35 mM NaCl, 10 mM histidine/histidine-HCl, 1 mM MgCl₂, 0.1 mM EDTA, 7.5% (w/v) sucrose, 0.1% (w/v) PS 80, 0.5% (v/v) ethanol, pH 6.6). The formulated bulk was then filtered and chased using chase buffer (35 mM NaCl, 10 mM histidine/histidine-HCl, 1 mM MgCl₂, 0.1 mM EDTA, 7.5% (w/v) sucrose, 0.1% (w/v) PS 80, 0.5% (v/v) ethanol, pH 6.6) into an intermediate holding vessel, after which it was stored at 2-8° C. in CryoVault containers or Allegro bags prior to freeze.

The material filled in the final storage container is designated Drug Substance.

### Freeze

The freeze step is used to freeze the Drug Substance prior to long term storage at −90 to −55° C. The upper end of the long term storage range (−55° C.) has been set to avoid the product's glass transition temperature of approximately −43° C. while the lower end of the range (−90° C.) has been set to accommodate freezer variation. The freeze step was carried out in either the CryoVault containers or Allegro bags final storage containers.

The CryoVault containers were frozen in a controlled manner using the Farrar Blast Freezer or Klinge Freezer. For the Farrar Blast Freezer, the temperature is set to −80° C. and the containers are frozen for at least 14 hours. For the Klinge Freezer, the temperature is set to −65° C. and the containers are frozen for at least 20 hours. Alternatively, freeze of CryoVault containers can be carried out in a passive manner using a general lab freezer set to −65° C. for at least 20 hours. The Allegro bags with RoSS units were frozen in a controlled manner using the RoSS.pFTU equipment set to −80° C. for at least 12 hours.

The yields obtained for Process B run at 1000 L are provided in Table 3, and quality measures obtained for Process B run at 1000 L are provided in Table 4.

TABLE 3
Process B yield when run at 1000 L

| Step | Weight (kg) | Titer (gc/mL) | Yield (%) |
|---|---|---|---|
| Bulk Virus Harvest | 758.5 | 2.08E+11 | N/A |
| Lysate | 844.5 | 2.02E+11 | 108 |
| Clarified Lysate^{a} | 961 | 1.51E+11 | 85 |
| Sartobind Q | 102 | 1.20E+12 | 92 |
| Unformulated Bulk | 35 | 3.86E+12 | 110 |
| Formulated Bulk^{b} | 34.7 | 4.32E+12 | 108 |
| Drug Substance | 28.7 | 2.70E+12 | 75 |

^{a}100 L clarified lysate removed;
^{b}10 L formulated bulk removed

TABLE 4
Process B product quality when run at 1000 L

| | | | VP Conc. | | | A260/A280 | | | | | | |
|---|---|---|---|---|---|---|---|---|---|---|---|---|
| | | VP Conc. | (AEX- | | A260/A280 | Ratio | | | | | | |
| | I, | (VP_260), | HPLC), | GC Conc. | Ratio | (AEX | | | PS 20 | PS 80 | Pluronic | |
| Step | ifu/mL | vp/mL | vp/mL | (gc/mL) | (UV_VIS) | HPLC) | VG:I | VP:I | Conc | Conc | F68 | Silicone |
| Lysate | NA | NA | NA | NA | NA | NA | NA | NA | NA | NA | NA | NA |
| Clarified | 3.2 × 10⁹ | NA | NA | 1.5 × 10¹¹ | NA | NA | 47 | NA | 1% | NA | 2600 μg/mL | 53 ppm |
| Lysate | | | | | | | | | | | | |
| Sartobind | 1.6 × 10¹⁰ | 1.6 × 10¹² | NA | 1.2 × 10¹² | 1.1 | NA | 75 | 100 | <LOQ | NA | 19 μg/mL | 11 ppm |
| Q Product | | | | | | | | | | | | |
| Ultrafiltered | 4.7 × 10¹⁰ | 4.0 × 10¹² | 3.5 × 10¹² | 3.9 × 10¹² | 1.1 | 1.3 | 82 | 85 | NA | NA | NA | NA |
| Product | | | | | | | | | | | | |
| Formulated | 4.8 × 10¹⁰ | 5.3 × 10¹² | 3.4 × 10¹² | 4.2 × 10¹² | 1.2 | 1.3 | 88 | 111 | NA | 0.08 | NA | NA |
| Product | | | | | | | | | | | | |
| Drug | 4.5 × 10¹⁰ | 3.3 × 10¹² | 2.7 × 10¹² | 2.7 × 10¹² | 1.1 | 1.3 | 60 | 74 | NA | 0.08 | NA | NA |
| Substance | | | | | | | | | | | | |

I—Infectivity;
VP—Viral Particle;
GC—Gene copy;
VG:I—Viral Genome:Infectivity;
VP:I—Viral Particle:Infectivity;
PS—Polysorbate

### Example 3: Comparison of Process A and Process B

As shown in Table 5, use of Process B resulted in an approximately 3-fold yield improvement and equivalent product quality compared to Process A. Furthermore, as will be readily appreciated, Process B requires fewer raw materials, e.g. due to the removal of the tangential flow filtration and bioburden removal 1 step. In addition, comparable solution volumes are used for Process 3 and Process 4 even though the cycle number is 2× higher for Process 4 than Process 3.

TABLE 5
Comparison of Process A and Process B.

| | | | | DNA Protein | | qPCR Genome |
|---|---|---|---|---|---|---|
| | Osmolality | | VP/mL by | Ratio by AEX | Infectivity | Concentration |
| Process | (mOsmol/kg) | PS 80% | AEX | (A260/A280) | (IFU/mL) | (GC/mL) |
| A | 419 | 0.1202 | 1.1 × 10¹¹ | 1.3 | 9.85 × 10⁸ | 7.09 × 10¹⁰ |
| A | 419 | 0.1185 | 1.9 × 10¹¹ | 1.3 | 18.61 × 10⁸ | 1.36 × 10¹¹ |
| A | 421 | ND | ND | ND | ND | ND |
| A | 421 | ND | ND | ND | ND | ND |
| A | 417 | ND | ND | ND | ND | ND |
| A | 420 | ND | ND | ND | ND | ND |
| B | 410 | 0.0915 | 5.5 × 10¹¹ | 1.3 | 94.55 × 10⁸ | 3.66 × 10¹¹ |

PS—polysorbate; VP—viral particle; AEX—anion exchange chromatography; ND—not defined.

### Example 4: Adenovirus Purification—Clarification Step of Process B

Two depth filtration trains can be utilized during the clarification step of Process B, prior to the 0.2 μm filtration step. The primary method is the use of Millistak+® HC Pro Pod depth filters having COSP media (“COSP filter”) alone and is as described above in Example 2. The secondary method is the use of COSP filters followed in series by Millistak+® Pod depth filters having XOHC media (“XOHC filter”) or Millistak+® HC Pro Pod depth filters having XOSP media (“XOSP filter”).

For the secondary method, the depth filters were first flushed with water for injection and then equilibrated with equilibration buffer (50 mM tris, 2 mM MgCl₂, 5% (w/v) sucrose, 1% (v/v) PS 20, pH 8.0) prior to initial loading. Benzonase®-treated lysate was then loaded onto the membrane. Loading targeted a 3 COSP:1 XOHC or XOSP ratio for optimum load distribution. After loading, chase buffer (50 mM tris, 2 mM MgCl₂, 5% (w/v) sucrose, 1% (v/v) PS 20, pH 8.0) was added to the membrane. Product collection began after 75% of filter hold up volume was diverted to waste in order to reduce product dilution and ended at the conclusion of chase.

As shown in Table 6, use of the different filtration steps results in similar filtrate titers and similar host cell protein clearance.

TABLE 6
Comparison of depth filters in clarification step

| | qPCR Titer | AEX Titer | HCP | HCP |
|---|---|---|---|---|
| Filter Train | (GC/mL) | (vp/mL) | (ng/mL) | (ng/dose) |
| C0SP + SHC | 1.7 × 10¹¹ | 2.4 × 10¹¹ | 81,196 | 16,606 |
| C0SP + X0SP + SHC | 1.5 × 10¹¹ | 2.4 × 10¹¹ | 80,574 | 16,864 |
| C0SP + X0HC + SHC | 1.6 × 10¹¹ | 2.5 × 10¹¹ | 78,806 | 16,023 |

HCP-host cell protein

### Example 5 Adding Mixed Mode Size Exclusion Chromatography Step—Variation of Process B

The mixed mode size exclusion step is designed to bind process-related impurities while allowing the product to flow through the column. The mixed mode size exclusion column was first equilibrated with 3 column volumes of equilibration buffer (50 mM tris, 222 mM NaCl, 1 mM MgCl2, 5% (w/v) sucrose), pH 8.0 prior to initiating the load. The adjusted Sartobind Q pool is loaded directly to the column with a target load volume of >/=60 L Sartobind Q pool/L resin at a target residence time of >/=4 minutes. After loading, the column is equilibrated 3 column volumes of equilibration buffer 50 mM tris, 222 mM NaCl, 1 mM MgCl2, 5% (w/v) sucrose), pH 8.0. The column is regenerated with 3 column volumes of strip buffer (1 M NaOH, 30% Isopropyl alcohol). Finally, the column is stored by washing with 3 column volumes of either 0.1 N NaOH or 20% Ethanol.

The use of mixed mode size exclusion chromatography step (Capto Core 700) reduced host cell protein concentrations from >100 ng/dose to less than 20 ng/dose

Mixed mode size exclusion chromatography improved Tangential Flow Filtration performance and reduced permeate flux decay by at least 29% and membrane fouling by 62%.

1. A method of purifying adenovirus from an adenovirus-containing sample comprising or derived from a host cell population having a cell density of at least about 4×106 cells/mL, the method comprising:
(a) clarifying the sample to provide a clarified sample, wherein clarification comprises depth filtration followed by microfiltration;
(b) processing the clarified sample by anion exchange chromatography to provide an anion exchange product; and
(c) processing the anion exchange product by tangential flow filtration (TFF) to provide a TFF product, wherein TFF comprises ultrafiltration and diafiltration.

2. The method of claim 1, wherein the anion exchange product is processed in a further step by mixed mode size exclusion chromatography to provide a mixed mode size exclusion product and wherein the mixed mode exclusion product is processed by TFF of step (c).

3. The method of claim 1, wherein the adenovirus-containing sample comprises a host cell population.

4. (canceled)

5. The method of claim 1, wherein the method comprises lysing the host cell population prior to step (a) to provide a cell lysate.

6-11. (canceled)

12. The method of claim 1, wherein the adenovirus-containing sample comprises a cell lysate.

13-18. (canceled)

19. The method of claim 1, wherein the depth filtration in step (a) comprises using a single type of depth filter.

20. The method of claim 19, wherein the depth filter has a nominal filter rating of between about 0.2 μm and about 2 μm, optionally wherein the depth filter is a Millistak+® HC Pro Pod depth filter, COSP media.

21. The method of claim 1, wherein the depth filtration in step (a) comprises using at least two different depth filters in series.

22-24. (canceled)

25. The method of claim 1, wherein the microfiltration membrane has a membrane pore size of about 0.2 μm.

26. (canceled)

27. The method of claim 1, wherein the anion exchange chromatography in step (b) comprises applying the clarified sample to an anion exchange membrane.

28-31. (canceled)

32. The method of claim 1, wherein step (b) comprises processing the clarified sample by microfiltration prior to anion exchange chromatography.

33. The method of any one of the preceding claims, wherein step (b) comprises processing the anion exchange product by microfiltration.

34. (canceled)

35. (canceled)

36. The method of claim 1, wherein step (c) comprises processing the product from step (b) by depth filtration, optionally wherein the depth filter has a nominal filter rating of up to about 0.1 μm, e.g. a Millistak+® Pod depth filter, XOHC media or a Millistak+® HC Pro Pod depth filter, XOSP media.

37. The method of claim 1, wherein the ultrafiltration in step (c) comprises using an ultrafiltration membrane having a nominal molecular weight cutoff (NMWCO) between about 100 and 1000 kDa, between about 200 and 700 kDa, or between about 300 and 500 kDa.

38. (canceled)

39. (canceled)

40. The method of claim 1, wherein the diafiltration in step (c) comprises diafiltration with diafiltration buffer.

41. (canceled)

42. The method of any one of the preceding claims, wherein the TFF product comprises host cell proteins at a concentration of 200 ng or less per 0.5×10¹¹ adenovirus particles.

43. The method of claim 1, wherein the TFF product comprises host cell DNA at a concentration of 10 ng or less per 0.5×10¹¹ adenovirus particles.

44. The method of claim 1, wherein the TFF product has an infectivity of greater than or equal to about 2.4×10⁶ ifu/mL.

45. The method of claim 1, wherein the method further comprises formulating the TFF product to provide a formulated product.

46. The method of claim 1, wherein the method further comprises subjecting the TFF product or formulated product to sterile filtration to provide a drug substance.

47-57. (canceled)

58. The method of claim 1, wherein the host cell population consists of mammalian cells, e.g. T-REx cells.

59. (canceled)

60. The method of claim 1, wherein the adenovirus is a replication-deficient simian adenovirus.

61. (canceled)

62. (canceled)

63. (canceled)

64. The method of claim 1, wherein the adenovirus encodes nCov-19 spike protein.

65. A purified adenovirus obtainable by or obtained by the method of claim 1.

66. (canceled)


SEQUENCE LISTING (2 sequences)

SEQ ID NO: 1 - protein, 1273 aa, Severe acute respiratory syndrome-related coronavirus
MFVFLVLLPLVSSQCVNLTTRTQLPPAYTNSFTRGVYYPDKVFRSSVLHSTQDLFLPFFSNVTWFHAIHVSGTNGTKRFDNPVLPFNDGVYFASTEKSNIIRGWIFGTTLDSKTQSLLIVNNATNVVIKVCEFQFCNDPFLGVYYHKNNKSWMESEFRVYSSANNCTFEYVSQPFLMDLEGKQGNFKNLREFVFKNIDGYFKIYSKHTPINLVRDLPQGFSALEPLVDLPIGINITRFQTLLALHRSYLTPGDSSSGWTAGAAAYYVGYLQPRTFLLKYNENGTITDAVDCALDPLSETKCTLKSFTVEKGIYQTSNFRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKKSTNLVKNKCVNFNFNGLTGTGVLTESNKKFLPFQQFGRDIADTTDAVRDPQTLEILDITPCSFGGVSVITPGTNTSNQVAVLYQDVNCTEVPVAIHADQLTPTWRVYSTGSNVFQTRAGCLIGAEHVNNSYECDIPIGAGICASYQTQTNSPRRARSVASQSIIAYTMSLGAENSVAYSNNSIAIPTNFTISVTTEILPVSMTKTSVDCTMYICGDSTECSNLLLQYGSFCTQLNRALTGIAVEQDKNTQEVFAQVKQIYKTPPIKDFGGFNFSQILPDPSKPSKRSFIEDLLFNKVTLADAGFIKQYGDCLGDIAARDLICAQKFNGLTVLPPLLTDEMIAQYTSALLAGTITSGWTFGAGAALQIPFAMQMAYRFNGIGVTQNVLYENQKLIANQFNSAIGKIQDSLSSTASALGKLQDVVNQNAQALNTLVKQLSSNFGAISSVLNDILSRLDKVEAEVQIDRLITGRLQSLQTYVTQQLIRAAEIRASANLAATKMSECVLGQSKRVDFCGKGYHLMSFPQSAPHGVVFLHVTYVPAQEKNFTTAPAICHDGKAHFPREGVFVSNGTHWFVTQRNFYEPQIITTDNTFVSGNCDVVIGIVNNTVYDPLQPELDSFKEELDKYFKNHTSPDVDLGDISGINASVVNIQKEIDRLNEVAKNLNESLIDLQELGKYEQYIKWPWYIWLGFIAGLIAIVMVTIMLCCMTSCCSCLKGCCSCGSCCKFDEDDSEPVLKGVKLHYT

SEQ ID NO: 2 - protein, 1304 aa, Severe acute respiratory syndrome-related coronavirus
MDAMKRGLCCVLLLCGAVFVSASQEIHARFRRFVFLVLLPLVSSQCVNLTTRTQLPPAYTNSFTRGVYYPDKVFRSSVLHSTQDLFLPFFSNVTWFHAIHVSGTNGTKRFDNPVLPFNDGVYFASTEKSNIIRGWIFGTTLDSKTQSLLIVNNATNVVIKVCEFQFCNDPFLGVYYHKNNKSWMESEFRVYSSANNCTFEYVSQPFLMDLEGKQGNFKNLREFVFKNIDGYFKIYSKHTPINLVRDLPQGFSALEPLVDLPIGINITRFQTLLALHRSYLTPGDSSSGWTAGAAAYYVGYLQPRTFLLKYNENGTITDAVDCALDPLSETKCTLKSFTVEKGIYQTSNFRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKKSTNLVKNKCVNFNFNGLTGTGVLTESNKKFLPFQQFGRDIADTTDAVRDPQTLEILDITPCSFGGVSVITPGTNTSNQVAVLYQDVNCTEVPVAIHADQLTPTWRVYSTGSNVFQTRAGCLIGAEHVNNSYECDIPIGAGICASYQTQTNSPRRARSVASQSIIAYTMSLGAENSVAYSNNSIAIPTNFTISVTTEILPVSMTKTSVDCTMYICGDSTECSNLLLQYGSFCTQLNRALTGIAVEQDKNTQEVFAQVKQIYKTPPIKDFGGFNFSQILPDPSKPSKRSFIEDLLFNKVTLADAGFIKQYGDCLGDIAARDLICAQKFNGLTVLPPLLTDEMIAQYTSALLAGTITSGWTFGAGAALQIPFAMQMAYRFNGIGVTQNVLYENQKLIANQFNSAIGKIQDSLSSTASALGKLQDVVNQNAQALNTLVKQLSSNFGAISSVLNDILSRLDKVEAEVQIDRLITGRLQSLQTYVTQQLIRAAEIRASANLAATKMSECVLGQSKRVDFCGKGYHLMSFPQSAPHGVVFLHVTYVPAQEKNFTTAPAICHDGKAHFPREGVFVSNGTHWFVTQRNFYEPQIITTDNTFVSGNCDVVIGIVNNTVYDPLQPELDSFKEELDKYFKNHTSPDVDLGDISGINASVVNIQKEIDRLNEVAKNLNESLIDLQELGKYEQYIKWPWYIWLGFIAGLIAIVMVTIMLCCMTSCCSCLKGCCSCGSCCKFDEDDSEPVLKGVKLHYT